SEQUENCE LISTING (28 sequences)

SEQ ID NO: 1 - DNA, 696 bp, Artificial
  SAV2095
atgaagaaaacattactcgcatcatcattagcagtaggtttaggaatcgtagcaggaaatgcaggtcacgaagcccatgcaagtgaagcggacttaaataaagcatctttagcgcaaatggcgcaatcaaatgatcaaacattaaatcaaaaaccaattgaagctggggcttataattatacatttgactatgaagggtttacttatcactttgaatcagatggtacacactttgcttggaattaccatgcaacaggtgctaatggagcaaacatgagtgcacaagcacctgcaactaataatgttgaaccatcagctgttcaagctaatcaagtacaatcacaagaagttgaagcaccacaaaatgctcaaactcaacaaccacaagcatcaacatcaaacaattcacaagttactgcaacaccaactgaatcaaaagcatcagaaggttcatcagtaaatgtgaatgctcatctaaaacaaattgctcaacgtgaatcaggtggcaatattcatgctgtaaatccaacatcaggtgcagctggtaagtatcaattcttacaatcaacttgggattcagtagcacctgctaaatataaaggtgtatcaccagcaaatgctcctgaaagtgttcaagatgccgcagcagtaaaattatataacactggtggcgctggacattgggttactgcataa

SEQ ID NO: 2 - DNA, 696 bp, Artificial
  SAS1999
atgaagaaaacattactcgcatcatcattagcagtaggtttaggaatcgtagcaggaaatgcaggtcacgaagcccatgcaagtgaagcggacttaaataaagcatctttagcgcaaatggcgcaatcaaatgatcaaacattaaatcaaaaaccaattgaagctggggcttataattatacatttgactatgaagggtttacttatcactttgaatcagatggtacacactttgcttggaattaccatgcaacaggtactaatggagcagacatgagtgcacaagcacctgcaactaataatgttgcaccatcagctgttcaagctaatcaagtacaatcacaagaagttgaagcaccacaaaatgctcaaactcaacaaccacaagcatcaacatcaaacaattcacaagttactgcaacaccaactgaatcaaaatcatcagaaggttcatcagtaaatgtgaatgctcatctaaaacaaattgctcaacgtgaatcaggtggcaatattcatgctgtaaatccaacatcaggtgcagctggtaagtatcaattcttacaatcaacttgggattcagtagcacctgctaaatataaaggtgtatcaccagcaaatgctcctgaaagtgttcaagatgccgcagcagtaaaattatataacactggtggcgctggacattgggttactgcataa

SEQ ID NO: 3 - DNA, 696 bp, Artificial
  SACOL2088
atgaagaaaacattactcgcatcatcattagcagtaggtttaggaatcgtagcaggaaatgcaggtcacgaagcccatgcaagtgaagcggacttaaataaagcatctttagcgcaaatggcgcaatcaaatgatcaaacattaaatcaaaaaccaattgaagctggggcttataattatacatttgactatgaagggtttacttatcactttgaatcagatggtacacactttgcttggaattaccatgcaacaggtactaatggagcagacatgagtgcacaagcacctacaactaataatgttgcaccatcagctgttcaagctaatcaagtacaatcacaagaagttgaagcaccacaaaatgctcaaactcaacaaccacaagcatcaacatcaaacaattcacaagttactgcaacaccaactgaatcaaaatcatcagaaggttcatcagtaaatgtgaatgctcatctaaaacaaattgctcaacgtgaatcaggtggcaatattcatgctgtaaatccaacatcaggtgcagctggtaagtatcaattcttacaatcaacttgggattcagtagcacctgctaaatataaaggtgtatcaccagcaaatgctcctgaaagtgttcaagatgccgcagcagtaaaattatataacactggtggcgctggacattgggttactgcataa

SEQ ID NO: 4 - DNA, 696 bp, Artificial; identical to SEQ ID NO: 2
  MW2020

SEQ ID NO: 5 - DNA, 696 bp, Artificial; identical to SEQ ID NO: 2
  SAUSA300_2051

SEQ ID NO: 6 - DNA, 696 bp, Artificial
  SAR2184
atgaagaaaacattactcgcatcatcattagcagtaggtttaggaatcgtagcaggaaatgcaggtcacgaagcccaagcaagtgaagcggacttaaataaagcatctttagcgcaaatggcgcaatcaaatgatcaaacattaaatcaaaaaccaattgaagctggcgcttataattatacatttgactatgaagggtttacttatcactttgaatcagatggtacacactttgcttggaattaccatgcaacaggtgctaatggagcagacatgagtgcacaagcacctgcaactaataatgttgcaccatcagctgatcaatctaatcaagtacaatcacaagaagttgaagcaccacaaaatgctcaaactcaacaaccacaagcatcaacatcaaacaattcacaagttactgcaacaccaactgaatcaaaagcatcagaaggttcatcagtaaatgtgaatgatcatctaaaacaaattgctcaacgtgaatcaggtggcaatattcatgctgtaaatccaacatcaggtgcagctggtaagtatcaattcttacaatcaacttgggattcagtagcacctgctaaatataaaggtgtatcaccagcaaatgctcctgaaagtgttcaagatgccgcagcagtaaaactatataacactggtggcgctggacattgggttactgcataa

SEQ ID NO: 7 - DNA, 696 bp, Artificial
  SAB1980c
atgaagaaaacattactcgcatcatcattagcagtaggtttaggaatcgtagcaggaaatgcaggtcacgaagcccatgcaagtgaagcggacttaaataaagcatctttagcgcaaatggcgcaatcaaatgatcaaacattaaatcaaaaaccaattgaagctggcgcttataattatacatttgactatgaagggtttacttatcactttgaatcagatggtacacattttgcttggaattaccatgcaacaggtgctaatggagcagacatgagtgcacaagcacctgcaactaataatgttgcaccatcagctgatcaagctaatcaagtacaatcacaagaagttgaagcaccacaaaatgctcaaactcaacaaccacaagcatcaacatcaaacaattcacaagttactgcaacaccaactgaatcaaaagcatcagaaggttcatcagtaaatgtgaatgatcatctaaaacaaattgctcaacgtgaatcaggtggcaatattcatgctgtaaatccaacatcaggtgcagctggtaagtatcaattcttacaatcaacttgggattcagtagcacctgctaaatacaaaggtgtatcaccagcaaatgctcctgaaagtgttcaagatgccgcagcagtaaaattatataacactggtggcgctggacattgggttactgcataa

SEQ ID NO: 8 - DNA, 696 bp, Artificial; identical to SEQ ID NO: 1
  SA1898

SEQ ID NO: 9 - protein, 231 aa, Artificial
  SAS1999
MKKTLLASSLAVGLGIVAGNAGHEAHASEADLNKASLAQMAQSNDQTLNQKPIEAGAYNYTFDYEGFTYHFESDGTHFAWNYHATGTNGADMSAQAPATNNVAPSAVQANQVQSQEVEAPQNAQTQQPQASTSNNSQVTATPTESKSSEGSSVNVNAHLKQIAQRESGGNIHAVNPTSGAAGKYQFLQSTWDSVAPAKYKGVSPANAPESVQDAAAVKLYNTGGAGHWVTA

SEQ ID NO: 10 - protein, 231 aa, Artificial; identical to SEQ ID NO: 9
  SAOUHSC_02333

SEQ ID NO: 11 - protein, 231 aa, Artificial; identical to SEQ ID NO: 9
  MW2020

SEQ ID NO: 12 - protein, 231 aa, Artificial; identical to SEQ ID NO: 9
  SAUSA300_2051

SEQ ID NO: 13 - protein, 231 aa, Artificial
  SAV2095
MKKTLLASSLAVGLGIVAGNAGHEAHASEADLNKASLAQMAQSNDQTLNQKPIEAGAYNYTFDYEGFTYHFESDGTHFAWNYHATGANGANMSAQAPATNNVEPSAVQANQVQSQEVEAPQNAQTQQPQASTSNNSQVTATPTESKASEGSSVNVNAHLKQIAQRESGGNIHAVNPTSGAAGKYQFLQSTWDSVAPAKYKGVSPANAPESVQDAAAVKLYNTGGAGHWVTA

SEQ ID NO: 14 - protein, 231 aa, Artificial; identical to SEQ ID NO: 13
  SaurJH9_1582

SEQ ID NO: 15 - protein, 231 aa, Artificial; identical to SEQ ID NO: 13
  SaurJH1_1184

SEQ ID NO: 16 - protein, 231 aa, Artificial; identical to SEQ ID NO: 13
  SA1898

SEQ ID NO: 17 - protein, 231 aa, Artificial
  SACOL2088
MKKTLLASSLAVGLGIVAGNAGHEAHASEADLNKASLAQMAQSNDQTLNQKPIEAGAYNYTFDYEGFTYHFESDGTHFAWNYHATGTNGADMSAQAPTTNNVAPSAVQANQVQSQEVEAPQNAQTQQPQASTSNNSQVTATPTESKSSEGSSVNVNAHLKQIAQRESGGNIHAVNPTSGAAGKYQFLQSTWDSVAPAKYKGVSPANAPESVQDAAAVKLYNTGGAGHWVTA

SEQ ID NO: 18 - protein, 231 aa, Artificial
  SAB1980c
MKKTLLASSLAVGLGIVAGNAGHEAHASEADLNKASLAQMAQSNDQTLNQKPIEAGAYNYTFDYEGFTYHFESDGTHFAWNYHATGANGADMSAQAPATNNVAPSADQANQVQSQEVEAPQNAQTQQPQASTSNNSQVTATPTESKASEGSSVNVNDHLKQIAQRESGGNIHAVNPTSGAAGKYQFLQSTWDSVAPAKYKGVSPANAPESVQDAAAVKLYNTGGAGHWVTA

SEQ ID NO: 19 - protein, 231 aa, Artificial
  SAR2184
MKKTLLASSLAVGLGIVAGNAGHEAQASEADLNKASLAQMAQSNDQTLNQKPIEAGAYNYTFDYEGFTYHFESDGTHFAWNYHATGANGADMSAQAPATNNVAPSADQSNQVQSQEVEAPQNAQTQQPQASTSNNSQVTATPTESKASEGSSVNVNDHLKQIAQRESGGNIHAVNPTSGAAGKYQFLQSTWDSVAPAKYKGVSPANAPESVQDAAAVKLYNTGGAGHWVTA

SEQ ID NO: 20 - DNA, 23 bp, Artificial
  SAV2095 forward primer
gaagttgaagcaccacaaaatgc

SEQ ID NO: 21 - DNA, 23 bp, Artificial
  SAV2095 reverse primer
tgttgatgcttgtggttgttgag

SEQ ID NO: 22 - DNA, 20 bp, Artificial
  16S rRNA forward primer
tcgtgtcgtgagatgttggg

SEQ ID NO: 23 - DNA, 19 bp, Artificial
  16S rRNA reverse primer
gcttaagggttgcgctcgt

SEQ ID NO: 24 - DNA, 18 bp, Artificial
  Z sequence
actgaacctgaccgtaca

SEQ ID NO: 25 - DNA, 20 bp, Artificial
  sceD forward primer JD83
aatccaacatcaggtgcagc

SEQ ID NO: 26 - DNA, 20 bp, Artificial
  sceD reverse primer JD84
gcagtaacccaatgtccagc

SEQ ID NO: 27 - DNA, 18 bp, Artificial
  16S forward primer JD79
acgagcgcaacccttaag

SEQ ID NO: 28 - DNA, 18 bp, Artificial
  16S reverse primer JD80
ttcgctgccctttgtatt

(19) United States
(12) Patent Application Publication
(10) Pub. No.: US 2010/0099088 A1
(43) Pub. Date: Apr. 22, 2010

(54) RELIABLE DETECTION OF VANCOMYCIN-INTERMEDIATE STAPHYLOCOCCUS AUREUS

(75) Inventors: Jolyne Drummelsmith, Chance (DO); Marc Quellette, Sillery (CA)

Correspondence Address: GOODWIN PROCTER LLP;PATENT ADMINISTRATOR, 53 STATE STREET, EXCHANGE PLACE, BOSTON, MA, 02109-2881, US

(73) Assignee: Universite Laval

(21) Appl. No.: 12/445,200

(22) PCT Filed: Oct. 12, 2007
(86) PCT No.: PCT/CA2007/001852

§ 371 Date: Apr. 10, 2009

Related U.S. Application Data
(60) Provisional application No. 60/851,303, filed on Oct. 13, 2006.

(51) Int. Cl. C12Q 1/68; G01N 33/569

(52) U.S. Cl. 435/6; 435/7.33

(57) ABSTRACT

The present invention relates to a method for identifying a VISA strain in a sample comprising measuring SAV2095 expression levels, a kit and use of SAV2095 for same. The present invention also relates to a method for facilitating the development of an individualized treatment regimen comprising measuring the expression level of SAV2095. The present invention further relates to a method for monitoring the progression of a vancomycin treatment of an infection comprising measuring the expression level of SAV2095. The present invention also relates to the detection of Staphylococcus aureus in a sample.

### FIELD OF INVENTION

The present invention relates to the field of vancomycin-intermediate Staphylococcus aureus detection, for example, in a biological sample.

### BACKGROUND OF THE INVENTION

Staphylococcus aureus causes several severe diseases in humans, including bacterial endocarditis, pneumonia, and hospital- and community-acquired infections. One of the few remaining treatments for cases involving multidrug-resistant MRSA (methicillin-resistant S. aureus) is vancomycin (Vn). Treatment failures caused by S. aureus strains exhibiting intermediate levels of Vn resistance (Vn-intermediate S. aureus; VISA) occur globally [1-6]. Recently, VISA was found in a patient not exposed to glycopeptides [7] and Vn-resistant staphylococci have been found in healthy carriers [8].

The majority of instances of clinical S. aureus Vn resistance involve strains exhibiting intermediate levels of resistance (MIC ≧4-16 mg/L, although some strains displaying VISA characteristics may display higher levels of resistance). Such resistance generally develops during the course of Vn treatment and is not mediated by van gene acquisition as seen in enterococci [9] and a few highly-resistant clinical S. aureus isolates (VRSA) [10, 11]. Unlike most VISA, these strains exhibit a high level of vancomycin resistance (MICs of ≧32 mg/L). In the majority of instances of clinical vancomycin resistance involving S. aureus, the van genes are absent, suggesting that resistance has emerged due to point mutations of specific genes or regulatory elements.

The glycopeptide antibiotic vancomycin has an affinity for the D-alanyl-D-alanine peptide tail of the peptidoglycan (PG) precursor and binds these upon translocation of the lipid-linked monomer to the outer leaflet of the cell membrane [12]. The bulky vancomycin molecule creates a physical block, preventing access to and cleavage of the D-alanyl-D-alanine by transpeptidases, and also possibly interfering with the transglycosylation reaction [13]. This prevents the incorporation of new peptidoglycan monomers into the cell wall and, in the case of a dividing culture where cell wall synthesis is essential, leads to cell death.

Strong correlations between cell-wall thickness and resistance levels exist in numerous strains, both clinical and derived in vitro [7, 14-17]. A thicker cell wall appears to decrease Vn penetration through the cell wall and prevent its interaction with nascent precursors [18]. Structural changes in S. aureus PG have also been noted in some strains [16, 19-21] but not others [22, 23], and might explain in part the decreased autolysis and resistance to murein hydrolases exhibited by many VISA strains [16, 22, 24-27]. The expression levels of several penicillin-binding proteins (PBPs), such as PBP4 and PBP2, are altered in some in vitro-derived and clinically-resistant strains [17, 22, 26, 28-30]. While some genes and proteins have been implicated in intermediate vancomycin resistance, none of these have been shown to be universal to VISA to date.

Attempts to measure the prevalence of intermediate vancomycin resistance in clinical isolates of S. aureus have produced widely varying estimates between different hospitals in the same country as well as different countries (reviewed in [34]). Factors contributing to this variability include the difficulty of identifying resistant strains based on standard laboratory practices such as antibiotic diffusion tests. One such example is the Japanese strain Mu3 [35], which although isolated from a clinical case where vancomycin therapy failed, tests as susceptible to vancomycin (i.e. a MIC (minimum inhibitory concentration) for the drug of <4 mg/L). Efforts undertaken to resolve this inconsistency revealed the existence of subpopulations of cells able to grow at higher drug concentrations [35]. Several such strains have since been identified and are classified as heterogeneous VISA (hVISA).

The current standard for detection is the vancomycin agar screen test, where 10 uL of a 0.5 McFarland unit suspension of pure culture is spotted on a BHI plate containing 6 mg/L of vancomycin and incubated at 35° C. for 24 h. Growth of >1 colony is considered a positive result. While suitable for strains displaying MIC≧8 mg/L, it is not reliable for strains displaying MICs below that.

A potential biomarker for VISA has been proposed (patent application NO. WO 2004/099444), based on decreased expression of the fmtB/mrp gene and/or a mrp homologue in VISA versus vancomycin-sensitive strains. However, in this test a negative result (i.e. no detectable expression) is considered as a positive test, which is not a desirable situation as a negative result could also be indicative of a test failure.

Recently, several transcriptomic and proteomic studies have linked a protein of unknown function, homologous to Mu50 locus SAV2095, to vancomycin resistance. Both the transcript and recently the protein were found to be increased upon in vitro selection of VISA to VRSA by DNA microarray [31] and 2D gel analysis [32], and the RNA was 10.5 fold increased in a clinical VISA isolate versus its MRSA parent [33]. DNA and predicted protein sequence alignments for this ORF in a number of sequenced strains are given in FIG. 1 and FIG. 2. The gene encodes a signal peptide and putative lytic transglycosylase domain, which suggest that the protein product may be exported and might be involved in degradation of the cell wall peptidoglycan.

Although the study of Mongodin et al. has identified SAV2095 as being upregulated at the protein and RNA level in highly-resistant vancomycin S. aureus, Mongodin et al. did not observe such difference of expression between a vancomycin-susceptible strain and a VISA strain.

The literature concerning VISA resistance is also peppered with examples of genes or proteins modulated in some resistant strains but not others. Expression levels of two genes affecting Vn resistance, mprF [36, 37] and tcaA [38, 39], were recently shown to not correlate to resistance in multiple strains [40].

There is thus a need to identify genes which may be used as reliable biomarkers for the recognition of VISA strains in general. Such genes would allow the development of a rapid and accurate test for distinguishing VISA strains from non-VISA strains.

The present invention seeks to meet these needs and other needs.

### SUMMARY OF THE INVENTION

By measuring mRNA levels and protein expression product (levels) in a high number of vancomycin-sensitive (for e.g. MRSA) and VISA strains of varying degrees of genetic relatedness, a link between vancomycin resistance level and SEQ ID NO.:1 (SAV2095) expression level was discovered.

Indeed, it was found that the protein encoded by SEQ ID NO.:1 is upregulated in VISA in comparison with VSSA by 2D gel proteomic comparison of VISA and non-VISA clinical strains. Furthermore, the expression product of the gene SAV2095 (SEQ ID NO.:1) at the RNA level is also increased in numerous VISA (including hVISA) versus vancomycin-sensitive MRSA strains. The expression level of this gene is also consistently increased further in VISA strains upon growth in the presence of subinhibitory levels of vancomycin (i.e. 3 mg/L), whereas in MRSA, the expression level of SEQ ID NO.:1 in the presence of subinhibitory levels of vancomycin (i.e. 1 mg/L) does not consistently increase. It is therefore a new and surprising feature of the present invention that measurement of the expression level of SEQ ID NO.:1 may be used reliably to detect the presence of VISA or a VISA profile. None of the references discussing the upregulation of SAV2095 in VISA strains has shown that this gene is reliable biomarker which may be used in the general identification of VISA strains of different genetic background in a biological (clinical) sample. The present invention provides for improved clinical diagnostics of infections.

The present invention therefore provides a method for the identification of a vancomycin-intermediate Staphylococcus aureus (VISA) bacteria in a sample, for example, in a biological sample. The method may comprise, for example, measuring a SEQ ID NO.:1 expression product (e.g., RNA, protein, etc.) or a SEQ ID NO.:1 homolog expression product, a complement of SEQ ID NO.:1 expression product or homolog thereof or a portion of SEQ ID NO.:1 expression product or homolog thereof in a sample comprising or suspected of comprising a S. aureus bacteria. The method may not require sub-culture of the sample prior to identification. The method may not require prior selection of strains (VSSA, VISA) in vancomycin-containing medium.

The method for VISA identification may comprise measuring the expression product of SEQ ID NO.:1, in a sample, and may further comprise comparing the expression level with a positive and/or negative reference value (control expression level; control expression product).

In another aspect, the present invention provides a method for distinguishing a VISA strain from a non-VISA strain. The present invention more particularly relates in a further aspect to a method to distinguish a vancomycin-intermediate Staphylococcus aureus (VISA) bacteria from a vancomycin-sensitive Staphylococcus aureus bacteria. The method may comprise comparing the expression level of SEQ ID NO.:1 from a S. aureus bacteria with a reference value associated with a level of expression of SEQ ID NO.:1 in vancomycin-sensitive S. aureus bacteria (negative reference value), wherein a higher level measured for the sample in comparison with the negative reference value is indicative of a VISA strain. The reference value may be derived from at least one strain which are either closely or distantly genetically related (or both) to the test strain (in a sample). The present invention may advantageously be performed without knowing the genetic background of the test strain (in a sample). It is a surprising feature of the present invention that distantly genetically related strains may be used as a reference value for a test strain in a sample.

In another aspect, the present invention relates to a kit for identifying a VISA in a (biological) sample. The kit may comprise a reagent for measuring the expression product of SEQ ID NO.:1, portion, homolog or complement thereof. In an additional aspect, the present invention relates to a kit for distinguishing vancomycin-intermediate Staphylococcus aureus (VISA) bacteria from a non-VISA stain, for example, vancomycin-sensitive Staphylococcus aureus bacteria such as, for example, a MRSA.

In a further aspect, the present invention relates to a method for monitoring the progression of a vancomycin treatment of an infection, for example a S. aureus infection. The method may comprise measuring a) an expression product of SEQ ID NO.:1 or a SEQ ID NO.:1 homolog, b) a complement of a), and/or c) a portion of a) or b) or combination thereof.

It is to be understood herein that any of SEQ ID NO.:1 or SEQ ID NO.:1 homologues or SEQ ID NO.:1 portions or SEQ ID NO.:1 complements may be used to carry out the present invention. Exemplary homologues are shown in FIG. 1. Homologues may show at least 60% homology or at least 65%, or at least 70%, or at least 75%, or at least 80% or at least 90% homology to SEQ ID NO.:1. It is to be understood herein that a portion of SEQ ID NO.:1, which may be used, for example in identification of a VISA, is a portion allowing measurement of such strain. For example, a portion of a protein encoded by SEQ ID NO.:1 which may suitably be detected may be of about 9 amino acids to about 231 amino acids. A nucleotide portion encoded by SEQ ID NO.:1 which may suitably be detected may be of about 12 to 20 nucleotides up to about 696 nucleotides. Such portions of SEQ ID NO.1 may represent a portion corresponding to about 5% of total nucleotide and/or amino acid coding for SEQ ID NO.:1 expression product, or about 10%, or about 15%, or about 20%, or about 25%, or about 30%, or about 35%, or about 40%, or about 45%, or about 50%, or about 55%, or about 60%, or about 65%, etc.

In yet another aspect, the present invention relates to a method for detecting a Staphylococcus aureus strain in a sample. The method may comprise the steps of detecting the expression level by measuring the expression product of SEQ ID NO.:1, in a sample.

A further aspect of the present invention provides for the use of SEQ ID NO.:1, SEQ ID NO.1 homologue, a sequence substantially complementary to SEQ ID NO.:1 or SEQ ID NO.1 homologue, a portion thereof or an expression product of SEQ ID NO.:1, SEQ ID NO.1 homologue for the detection of a S. aureus strain.

In another aspect, the present invention relates to the use of SEQ ID NO.:1, SEQ ID NO.1 homologue, a sequence substantially complementary to SEQ ID NO.:1 or SEQ ID NO.1 homologue, a portion thereof or an expression product of SEQ ID NO.:1, SEQ ID NO.1 homologue for the detection of a VISA strain.

In yet another aspect, the present invention relate to the use of SEQ ID NO.:1, SEQ ID NO.1 homologue, a sequence substantially complementary to SEQ ID NO.:1 or SEQ ID NO.1 homologue, a portion thereof or an expression product of SEQ ID NO.:1, SEQ ID NO.1 homologue for the distinction of a VISA strain from a non-VISA strain.

### BRIEF DESCRIPTION OF THE DRAWINGS

In drawings which illustrates non-limitative exemplary embodiments of the present invention,

FIG. 1 is an alignment of DNA sequences of SAV2095 (SEQ ID NO.:1) homologues from various sequenced strains. Bases in black are identical in all sequences. Locus(strain) designations are as follows: SAS1999(MSSA476), SACOL2088(COL), MW2020(MW2), SAUSA300_{—}2051(USA300), SAR2184(MRSA252), SAB1980c(RF122), SAV2095(Mu50), SA1898(N315). Alignment was generated using CLUSTALW 1.8 and displayed using Boxshade;

FIG. 2 is an alignment of predicted protein sequences of SAV2095 homologues from various sequenced strains. Amino acids in black are identical in at least 70% of the sequences compared. A “*” in the consensus line indicates absolute identity, whereas a ‘.’ indicates homology. A blank space indicates a non-homologous substitution. Strains are as in FIG. 1 with the addition of SaurJH9_{—}1582 (strain JH9) and SaurJH1_{—}1184(strain JH1), which are temporary identifiers as the genome sequences have not yet been completed;

FIG. 3 is a 2D gel comparison of A, CMRSA-2 (MRSA) and B, Mu50 (VISA), showing the spot identified as SceD (locus SAV2095) by mass spectrometry (circled, with arrow in last panel of each);

FIG. 4 shows transmission electron micrographs of selected MRSA strains and in vitro-selected mutants with decreased Vn sensitivity. A, CMRSA-1 and mutant C1V8; B, CMRSA-2 and mutant C2V8; C, CMRSA-3 and mutant C3V8. Strains are as in Table 1. The cell wall thickness in nm and 95% CI is listed below each strain.

FIG. 5 is a 2D gel comparison of A, MRSA and B, hVISA/VISA strains;

FIG. 6 is a graph illustrating the expression of SAV2095 homologue in various MRSA (VSSA) and VISA strains by quantitative reverse-transcriptase polymerase chain reaction (qRT-PCR). Strain names are as in Table 1;

FIG. 7 shows relative SAV2095 mRNA expression levels in various MRSA, hVISA (MIC<4 mg/L) and VISA (MIC≧4 mg/L) strains as described in Table 1. The agr type and MLST of each strain is noted where this is known, highlighting the variety of genetic backgrounds tested;

FIG. 8 is a sceD (SAV2095) mRNA quantitation from blood spiked with VSSA and VISA strains;

FIG. 9 is a graph illustrating the effect of subinhibitory concentrations of vancomycin (Vn) on SAV2095 homologue expression level in various MRSA (VSSA) and VISA strains by qRT-PCR. For MRSA, growth occurred in the presence of 1 mg/L Vn, while for VISA strains 3 mg/L was used. Strain names are as in Table 1.

FIG. 10 shows SAV2095 mRNA expression in series of step-wise in vitro-selected Vn-resistant mutants. MIC by standard Etest is in brackets for each strain.

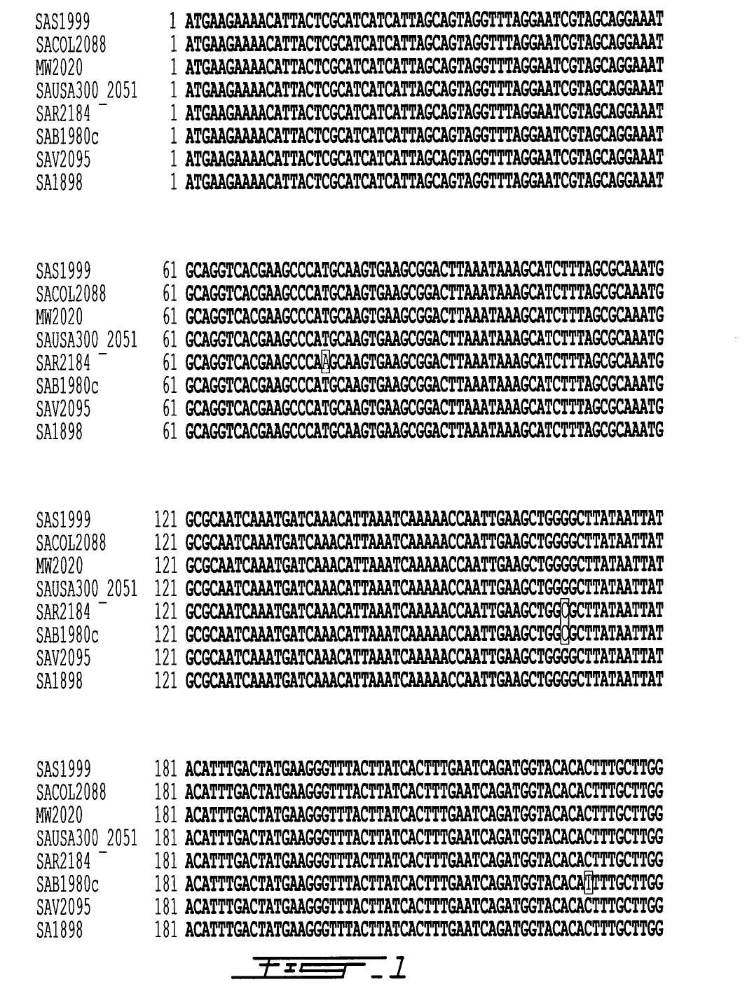

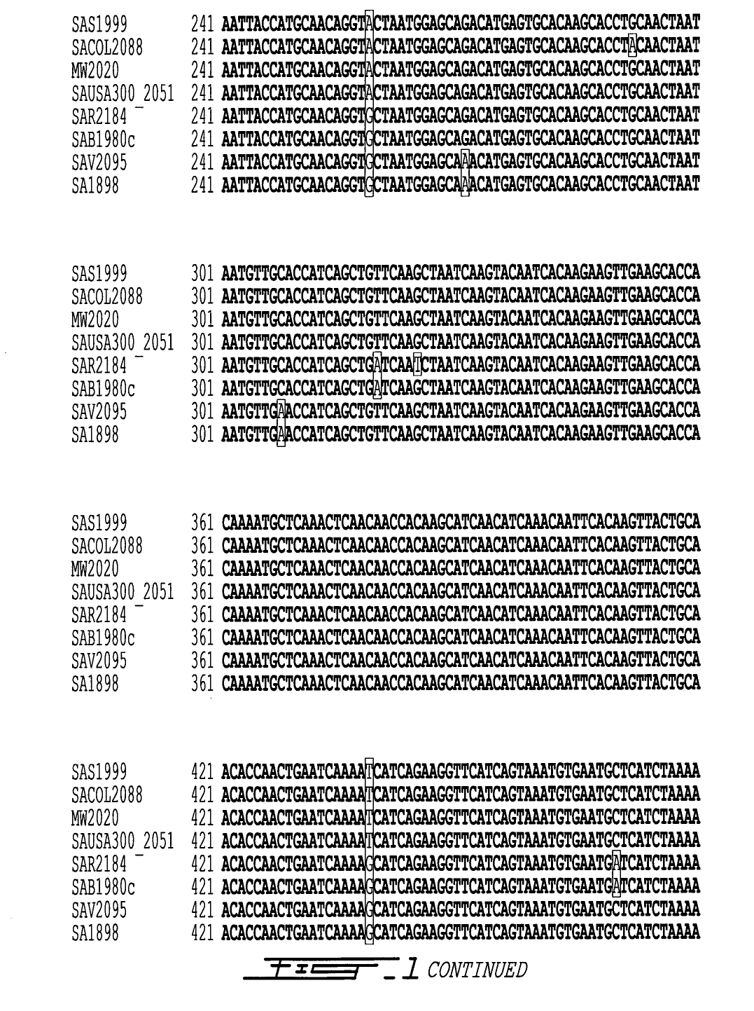

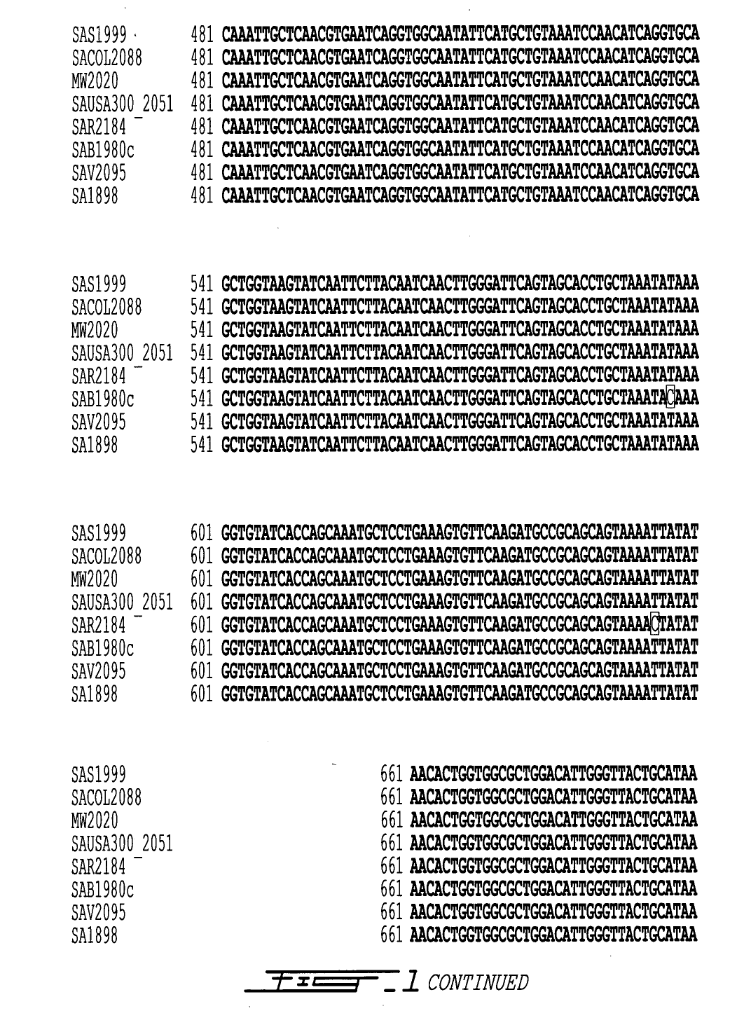

### DETAILED DESCRIPTION

In order to provide a clear and consistent understanding of the terms used in the present disclosure, a number of definitions are provided below. Moreover, unless defined otherwise, all technical and scientific terms as used herein have the same meaning as commonly understood to one of ordinary skill in the art to which this invention pertains.

As used in the specification and claim(s), the words ‘comprising’ (and any form of comprising, such as ‘comprise’ and ‘comprises’), ‘having’ (and any form of having, such as ‘have’ and ‘has’), ‘including’ (and any form of including, such as ‘include’ and ‘includes’) or ‘containing’ (and any form of containing, such as ‘contain’ and ‘contains’), are inclusive or open-ended and do not exclude additional, unrecited elements or process steps. The term “about” is used to indicate that a value includes an inherent variation.

The present invention relates in one aspect thereof to a method for identifying a VISA strain in a sample, for example and without limitation, in a biological sample. The method may comprise measuring an expression level of a) an expression product of SEQ ID NO.:1 or of a SEQ ID NO.:1 homolog, b) a complement of a), c) a portion of a) or b), and comparing the expression level with a reference value (a positive reference value, a negative reference value and/or both). As used herein “VISA” means Vancomycin Intermediate Staphylococcus aureus strain. VISA strains may also comprise hVISA strains. A non-VISA strain may be a vancomycin-sensitive Staphylococcus aureus (VSSA). A VSSA of the present invention may be a methicillin resistant Staphylococcus aureus (MRSA). Genetic relatedness of different strains may be evaluated by MLST. MLST (multi locus sequencing typing) refers to a nucleotide sequence based approach for characterizing isolates of bacteria (www.mlst.net) based on the sequencing of seven highly conserved housekeeping locus in bacteria. “Closely genetically related” means strains that share at least about 5 to 7 loci in common based on MLST typing. “Distantly genetically related” means strains that share about less than 5 loci in common based on MLST typing. The present invention relates to all VISA strains and relates to VISA of all MLST. As such, the present invention may suitably be carried out for strains other than MLST 5 and/or MLST105.

In an embodiment of the present invention, a SEQ ID NO.:1 expression product may be, for example, a polynucleotide (for example RNA) which may be transcribed (encoded) by SEQ ID NO.1, a homolog to a polynucleotide encoded by SEQ ID NO.:1, a complement to a polynucleotide encoded by SEQ ID NO.:1, a complement to a homolog of a polypeptide encoded by SEQ ID NO.:1, a portion of a polynucleotide encoded by SEQ ID NO.1, a portion of a homolog to a polypeptide encoded by SEQ ID NO.:1, and/or a portion of a complement to a polynucleotide encoded by SEQ ID NO.:1 or a complement to a homolog of a polypeptide encoded by SEQ ID NO.:1.

Any polynucleotide, polynucleotide homolog, polynucleotide complement and/or polynucleotide portion may be detected by several methods known in the art, including, without limitation, polymerase chain reaction (PCR), reverse transcriptase polymerase chain reaction (RT-PCR), Northern blot, Southern blot, hybridization with probes, etc. The scope of this invention is not limited to the use of amplification by any such technologies, but rather includes the use of any nucleic acid amplification method or any other procedure which may be used to increase the sensitivity and/or the rapidity of nucleic acid-based diagnostic tests. The scope of the present invention also covers the use of any nucleic acids amplification and detection technology including real-time or post-amplification detection technologies and any amplification technology combined with detection. Identification by any sequencing method is also under the scope of the present invention.

A reagent (detection reagent) for measuring such expression products (polynucleotides) may be a sequence substantially complementary to a polynucleotide encoded by SEQ ID NO.:1, a sequence substantially complementary to a homolog of a polypeptide encoded by SEQ ID NO.:1, a sequence substantially complementary to a complement of a polynucleotide encoded by SEQ ID NO.:1, a sequence substantially complementary to a complement of a homolog of a polynucleotide encoded by SEQ ID NO.:1, a sequence substantially complementary to a portion of a polynucleotide encoded by SEQ ID NO.1, a sequence substantially complementary to a portion of a homolog to a polypeptide encoded by SEQ ID NO.:1, and/or a sequence substantially complementary to a portion of a complement to a polynucleotide encoded by SEQ ID NO.:1 or a complement to a homolog of a polypeptide encoded by SEQ ID NO.:1.

As used herein the term “complementary” refers to a portion of a nucleic acid molecule that is capable of base pairing with another nucleic acid molecule with a perfect (e.g., 100%) match. “Substantially complementary” with respect to a nucleic acid means that the sequence has from about 70% to about 100% or about 80 to about 100% or about 85%, 90% or 95% to about 100% complementarity over a sequence or over a portion of a sequence. It is to be understood herein that a polynucleotide of the present invention (i.e., target), including SEQ ID NO.:1, SEQ ID NO.:1 homolog or any portions thereof may be detected using a nucleotide sequence (i.e., a probe or primer) including a portion which is substantially complementary to such polynucleotide. It is also to be understood herein that a complement of SEQ ID NO.:1, SEQ ID NO.:1 homolog or any portions thereof (i.e., target complement) may be detected using a nucleotide sequence (i.e., a probe or primer) including a portion which is substantially complementary to such complement. The length of the target which is sought to be detected may vary. For example, a target of at least 12, at least 15, at least 20 nucleotides or even the full length of the target may suitably be detected using a probe and/or primer. The length of the probe or primer used to detect a target may vary from about 8 to about 50 bases or more (or any sub-range, e.g., 15 to 50, 20 to 45, etc.), although other lengths may suitably be used without departing from the scope of the invention.

In a further embodiment, a SEQ ID NO.:1 expression product may be, for example, a polypeptide (protein, peptide) encoded by SEQ ID NO.:1, of an homolog of such polypeptide or fragments thereof.

A reagent (detection reagent) for measuring such expression products (polypeptides) may be a compound which is capable of specific binding to such polypeptides. Such compound may be linked to a reporter molecule (e.g., fluorochrome, enzyme, radioactive label, etc.) thereby allowing a signal to be detected following or upon binding. By “signal” is meant a characteristic change that is observable or measurable by physical, chemical, or biological means known to those skilled in the art. For example, a change in emission or absorbance of visible (for example colorimetric detection) or invisible light at a certain wavelength, electrical conductivity, emission of radioactivity, etc. Exemplary detection reagents (compounds) specifically binding to (recognizing) polypeptides are known in the art and may include an antibody, an antibody binding fragment, an engineered binding fragment (diantibody triantibody, minibody, single domain antibody, etc) and/or an aptamer. Alternatively, polypeptides may be detected (and/or measured) by their physical properties using, for example and without limitation, HPLC profiles, electrophoresis, two-dimensional gel electrophoresis (2DE) and/or mass spectrometry.

An antibody may be generated using methods that are well known in the art. Such antibodies may include, but are not limited to, polyclonal, monoclonal, chimeric, and single chain antibodies, Fab fragments, and fragments produced by a Fab expression library. Monoclonal antibodies (MAbs) may be made by one of several procedures available to one of skill in the art, for example, by fusing antibody producing cells with immortalized cells and thereby making a hybridoma. The general methodology for fusion of antibody producing B cells to an immortal cell line is well within the province of one skilled in the art. Another example is the generation of MAbs from mRNA extracted from bone marrow and spleen cells of immunized animals using combinatorial antibody library technology. In addition, techniques developed for the production of chimeric antibodies may be used. Alternatively, techniques described for the production of single-chain antibodies may be employed. Fabs that may contain specific binding sites for a polypeptide encoded by SEQ ID NO.:1, a homolog to a polypeptide encoded by SEQ ID NO.:1, a portion of a polypeptide encoded by SEQ ID NO.:1 and/or a portion of a homolog to a polypeptide encoded by SEQ ID NO.:1, may also be generated. Various immunoassays may be used to identify antibodies having the desired specificity. Numerous protocols for competitive binding or immunoradiometric assays using either polyclonal or monoclonal antibodies with established specificities are well known in the art. To obtain polyclonal antibodies, a selected animal (for example and without limitation, a rabbit, a chicken, etc) may be immunized with a protein or polypeptide derived from the polypeptide encoded by SEQ ID NO.:1 or from an homolog of a polypeptide encoded by SEQ ID NO.:1. Serum from the animal (or extraction from egg yolks in the event of chicken immunization) may be collected and treated according to known procedures. Polyclonal antibodies to the protein or polypeptide of interest may then be purified by affinity chromatography. Techniques for producing polyclonal antisera are well known in the art.

Measuring an expression level may comprise, for example, contacting a biological sample with a reagent for detecting (measuring) the expression product of SEQ ID NO.:1 as described above. For example, detecting may involve measuring a signal emitted following binding between SEQ ID NO.:1 expression product and a reagent capable of specific recognition of SEQ ID NO.:1 (for example and without limitation, a sequence substantially complementary and/or an antibody). It is to be understood herein that expression products may comprise the expression product of a DNA (gene) for example a RNA (for example mRNA) and/or a polypeptide or protein or any products therefrom (cDNA, siRNA, etc.)

In an embodiment of the present invention, a polypeptide or a polypeptide homolog or a portion of a polypeptide and/or a portion of a polypeptide homolog may be detected by immunoassay. By “immunoassay” it is meant a direct or indirect immunoassay. Such assay include without limitation competitive binding assay, non-competitive binding assay, radioimmunoassay, enzyme-linked immunosorbent assay (ELISA), sandwich assay, precipitin reaction, gel diffusion immunodiffusion assay, agglutination assay, fluorescence immunoassay, chemoluminescence immunoassay, immunoPCR immunoassay, protein A or protein G immunoassay and immunoelectrophoresis assay, etc.

In accordance with the present invention, the expression level of SEQ ID NO.:1 (or of any of the SEQ ID NO.:1 homolog, portion or complement thereof in the sample) may be compared with a reference value (of the same expression product). The method may comprise, for example, a step of comparing a level of expression of SEQ ID NO.:1 from a sample comprising or suspected of comprising a S. aureus bacteria with a reference value as described above. A higher level of SEQ ID No.:1 measured for the sample in comparison with the reference value derived from vancomycin-sensitive strain may be indicative of a VISA strain.

In accordance with the present invention, the reference value may be a positive reference value (control) and/or a negative reference value (control). A reference value obtained from at least one closely genetically related and/or distantly genetically related VISA strain may represent a positive reference value. A reference value obtained from at least one closely genetically related and/or distantly genetically related non-VISA strain, wherein the non-VISA may be a methicillin-resistant S. aureus (MRSA), may represent a negative reference value. In an embodiment of the present invention, the reference value may be measured from at least one or more than one (from at least two) closely genetically related and/or distantly genetically related S. aureus (VISA or non-VISA) strains. A reference value may be obtained from the average (mean) expression level of SEQ ID NO.:1 or homolog measured in closely genetically related and/or distantly genetically related S. aureus (VISA and/or non-VISA) strains. In accordance with the present invention, the reference value may be derived from about at least one or at least two vancomycin-sensitive S. aureus strains which are distantly genetically related to the suspected VISA (in the sample) and/or closely genetically related and/or distantly genetically related to one another. The reference value may be derived from strains which are distantly genetically related to the test strain (in a sample). A higher expression level measured in the sample in comparison with the negative reference value may be indicative of a VISA strain. An expression level equal to or higher than a positive value may be indicative of a VISA strain. An expression level equal or lower than a negative reference value and/or lower than a positive reference value may be indicative of a non-VISA strain. A non-detectable level of SEQ ID NO.:1 may be indicative of a non S. aureus strain. Of course, the mean average value may be normalized with the expression level of one or several (S. aureus) housekeeping genes, the expression of which is not affected by vancomycin. Such housekeeping genes may include, without limitation, the 16S ribosomal RNA or other genes whose expression is shown to be substantially stable among strains with various levels of Vn resistance, as determined, for example, using geNorm (42). Alternately, an exogenous RNA can be spiked into the sample at the extraction stage and used for normalization (43).

For example, a reference value may be obtained by isolating RNA from at least two VISA and/or VSSA strains, generating cDNA from this RNA, amplifying the cDNA and measuring the expression level of that cDNA by any means known in the art. An average may be calculated from the measured expression levels and such average may represent a negative reference value (in the case of VSSA) or a positive reference value (in the case of VISA). The positive and/or negative reference value may be predetermined and a kit may contain instructions whereby when the expression level in the sample is higher than a negative value (for example) a VISA strain may be identified. Alternatively, a kit may contain at least one strain known to be a VISA strain and/or a VSSA strain whereby the user determines and compares himself the expression level of SEQ ID NO.:1.

Alternatively, a reference value may be obtained by measuring the expression level of a polypeptide encoded by SEQ ID NO.:1 and/or a homolog to a polypeptide encoded by SEQ ID NO.:1 in at least two VISA and/or VSSA strains. Such measurement may be performed by using, for example, an antibody specific to a polypeptide encoded by SEQ ID NO.:1 and/or a homolog to a polypeptide encoded by SEQ ID NO.:1 and/or portions thereof. Detection of binding of the antibody to the expression product may be quantified by any means known in the art. An average may be calculated from the quantified expression levels and such average may represent a negative reference value (in the case of VSSA) or a positive reference value (in the case of VISA).

According to the present invention, a “sample” may be collected from any source, including without limitation, the environment (e.g., air, soil, dust, water, etc.), an object, a medical device (including, for example, catheters, sutures, artificial heart valves, central lines and/or anything indwelling or implanted in an individual), a food product, a pharmaceutical or cosmetic commodity, an animal, an individual, or any other source from which the identification of VISA is to be determined. In an embodiment of the present invention, a sample may be a “biological sample”. As used herein, a biological sample refers to a sample of biological fluids or tissues. It is also meant to encompass derivatives and fractions of such samples (e.g., cell lysates). A biological sample according to the present invention may be a specimen obtained from a patient suspected of having an infection, for example a Staphylococcus aureus bacteremia. The sample may also be obtained from a patient prior to or undergoing vancomycin therapy for a Staphylococcus aureus (methicillin-resistant) infection. A biological sample may be obtained from an individual directly or indirectly, including cells, tissue or fluid. Non-limiting examples of the sample may include blood, urine, semen, milk, sputum, mucus, a buccal swab, a nasal swab, a vaginal swab, a rectal swab, an aspirate, a needle biopsy, a section of tissue obtained for example by surgery or autopsy, plasma, serum, spinal fluid, lymph fluid, the external secretions of the skin, respiratory, intestinal, and genitourinary tracts, tears, saliva, tumors, organs, etc. The sample may be diluted, dissolved, suspended, extracted or otherwise treated to solubilize and/or purify any target (for example SEQ ID NO.:1) present or to render it accessible to reagents (detection reagents). Where the sample contains cells, the cells may be lysed or permeabilized to release the target from within the cells. The sample may be, for example, in a liquid, a solid and/or a semi-solid state. Detection of the SEQ ID NO.:1 expression product may be performed directly from the biological sample collected from an individual suspected of being infected or infected with S. aureus and/or using a culture (sub-culture) originating from the sample.

The present invention also relates to a diagnostic (detection) kit to identify the presence of a VISA in a biological sample and may comprise a reagent for identifying a VISA in a sample such as a reagent for measuring the expression level of a) an expression product of SEQ ID NO.:1 or a SEQ ID NO.:1 homolog, b) a complement of a), or c) a portion of a) or b) as described above. The kit may also comprise instructions for comparing the expression level of SEQ ID NO.:1 with a reference value indicative of the expression level of SEQ ID NO.:1 of at least one (or the average of at least two) (closely genetically related and/or distantly genetically related) vancomycin-sensitive Staphylococcus aureus strain. The kit may also comprise a container of known VISA and/or non-VISA strains to determine a positive and/or a negative reference value. The method may comprise the steps of mixing a reagent able to bind a SEQ ID NO.:1 expression product in a sample suspected of comprising a S. aureus bacteria and detecting the SEQ ID NO.:1 expression product by any means known in the art. The presence of the expression product may be indicative of the presence of a VISA strain and not a vancomycin-sensitive S. aureus strain. Of course the level of expression of SEQ ID NO.:1 in the sample may be compared with that of a standard which may be normalized to reflect the expression level expected for a vancomycin-sensitive S. aureus strain and/or a vancomycin-intermediate S. aureus strain.

The kit's reagent may be a sequence substantially complementary to SEQ ID NO.:1, a sequence substantially complementary to a SEQ ID NO.:1 homolog, a sequence substantially complementary to a complement of SEQ ID NO.:1, a sequence substantially complementary to a complement of a homolog of SEQ ID NO.:1, a sequence substantially complementary to a portion of SEQ ID NO.:1 and/or a sequence substantially complementary to a portion of a homolog of SEQ ID NO.:1.

The kit's reagent may be an antibody, an antibody binding fragment and aptamers.

In a further exemplary embodiment, the present invention relates to a method for facilitating the development of an individualized treatment regimen for a patient suspected of having an infection or in a patient having an infection in need of treatment, said method comprising measuring an expression level of a) an expression product of SEQ ID NO.:1 or a SEQ ID NO.:1 homolog, b) a complement of a), c) a portion of a) or b), comparing the expression level with a (positive and/or negative) reference value, and administering a vancomycin or non-vancomycin drug regimen in response to the result obtained whereby when SEQ ID NO.:1 or its expression product or a SEQ ID NO.:1 complement or a SEQ ID NO.:1 homologue is detected in a sample to a higher level than a negative reference value, vancomycin treatment is not provided to a patient or undergoing vancomycin treatment is stopped (terminated). When SEQ ID NO.1 is lower than a positive reference value, a vancomycin treatment may be administered. A non-vancomycin regimen, may include, without limitation, linezolid, quinupristin/dalfopristin, daptomycin, tigecycline rifampicin+fusidic acid, rifampicin+fluoroquinolone, pristinamycin, co-trimoxazole (trimethoprim-sulfamethoxazole), doxycycline, minocycline, clindamycin and/or gentamycin administration.

It is to be understood that the infection in need of treatment in a patient (individual) may be, for example a Staphylococcus aureus infection and/or a drug resistant Staphylococcus aureus infection. For example, the patient may have, or be suspected to have, a methicillin-resistant Staphylococcus aureus infection. The patient may have a Staphylococcus aureus infection that is susceptible to becoming resistant to vancomycin over time.

In yet a further aspect, the present invention relates to a method for monitoring the progression of a vancomycin treatment of an infection comprising measuring the expression level of a) an expression product of SEQ ID NO.:1 or a SEQ ID NO.:1 homolog, b) a complement of a), and/or c) a portion of a) or b) over the course of the treatment. The method may also comprise comparing the expression level with a reference value. For example, the infection may be a Staphylococcus aureus infection (methicillin resistant Staphylococcus aureus infection) for which a vancomycin treatment was initiated.

The present invention also relates to a method for identifying a Staphylococcus aureus strain in a sample. The method may comprise detecting an expression level of a) an expression product of SEQ ID NO.:1 or a SEQ ID NO.:1 homolog, b) a complement of a), c) a portion of a) or b).

### Materials and Methods

### MIC Determination

Vancomycin MICs were determined using standard Etest methods. Briefly, colonies from a fresh overnight plate were resuspended in saline to a turbidity equivalent to 0.5 McFarland units. This suspension was swabbed onto Mueller-Hinton agar plates, allowed to dry, and overlaid with a Vancomycin Etest strip (AB Biodisk). Plates were incubated at 37° C. for 48 h and interpreted. S. aureus strain ATCC29213 was used as a control (MIC=1.5 mg/L). For some MRSA strains, the macromethod Etest was also applied, using an inoculum equivalent to 2 McFarland units on Brain-Heart Infusion agar plates.

### Strains Used

The strains tested in this study, as well as vancomycin resistance level, multilocus sequence type (where known), and country of origin are presented in Table 1. These strains may be obtained from the A.T.C.C. or from the Network on antimicrobial resistance in S. aureus (see; www.narsa.net).

TABLE 1
STRAINS USED IN THIS STUDY

| | | | | | | |
|---|---|---|---|---|---|---|
| | Country of | | Modified | | Agar | |
| Name | Origin | Alias | Etest^{a} | CLSI Etest^{b} | screen^{c} | MLST |

MRSA strains

| | | | | | | |
|---|---|---|---|---|---|---|
| CMRSA-1 | Canada | 98S-329 | 8 I | 1.5 S | — | 45^{d} |
| CMRSA-2 | Canada | 98S-1241 | 3 S | 1.5 S | — | 5 |
| CMRSA-3 | Canada | 98S-1258 | 3 S | 2 S | — | 239 |
| CMRSA-4 | Canada | 98S-1237 | 2 S | 1 S | — | 36^{d} |
| CMRSA-5 | Canada | | — | — | — | |
| Hungarian | Hungary | HUSA304, | — | — | — | 239 |
| | | BAA-39 | | | | |
| New York | US-NY | NYBK2464, | — | — | — | 5 |
| | | BAA-41 | | | | |
| Brazilian | Portugal | HSJ216, BAA- | — | — | — | 239 |
| | | 43 | | | | |
| Sa 501V | | CCUG41787 | — | — | — | |
| F-182 | US-KN | ATCC43300 | — | — | — | |

hVISA/VISA strains

| | | | | | | |
|---|---|---|---|---|---|---|
| Mu50 | Japan | NRS1 | 12 I | 8 I | CG (24) | 5 |
| Mu3 | Japan | NRS2 | 6 I | 4 I | NG (72) | 5^{d} |
| SA MER | France | NRS11 | 8 I | 2 S | NG (72) | |
| SA MER-S20 | France | NRS14 | 24 I | 8 I | CG (24) | 5^{d} |
| HIP07930 | US-NY | NRS22 | 8 I | 4 I | IC (48) | 45^{d} |
| | | USA600 | | | | |
| | | 99758 | | | | |
| HIP09433 | US-MI | NRS27 | 8 I | 4 I | NG (72) | 45^{d} |
| HIP09662 | US-WV | NRS28 | 8 I | 3 S | NG (72) | |
| LIM 1 | France | NRS35 | 4 S | 2 S | NG (72) | 247 |
| LIM 3 | France | NRS37 | 6 I | 4 I | NG (72) | |
| BR 5 | Brazil | NRS56 | 16 I | 8 I | CG (24) | 239^{d} |
| LY-1999 | Oman | NRS63 | 16 I | 8 I | IC (48) | 372^{d} |
| 0620-01 | | | | | | |
| LY-1999 | Oman | NRS64 | 6 I | 1.5 S | NG (72) | |
| 0620-02 | | | | | | |
| NRS118 | US-CA | N/A | 24 I | 8 I | CG (24) | 247^{d} |
| 160013 | UK | NRS283 | 8 I | 4 I | IC (48) | 36 |
| HIP12864 | US-OK | NRS402 | 48 R | 8 I | CG (24) | |

in vitro strains Derived from

| | | |
|---|---|---|
| C1V4 | CMRSA-1 | 5 I |
| C1V8 | CMRSA-1 | 10 I |
| C1V30 | CMRSA-1 | 16 I |
| C2V4 | CMRSA-2 | 6 I |
| C2V8 | CMRSA-2 | 9 I |
| C2V24 | CMRSA-2 | 30 I |
| C3V4 | CMRSA-3 | 3 S |
| C3V8 | CMRSA-3 | 8 I |
| C3V24 | CMRSA-3 | 20 I |
| C4V8 | CMRSA-4 | 5 I |
| C4V20 | CMRSA-4 | 8 I |

^{a}Modified E-test (2 McF inoculum) results are reported as mg/L vancomycin; S = sensitive, I = intermediate, R = resistant. Values for MRSA were determined by us, for VISA are as reported by NARSA (www.narsa.net).
^{b}CLSI-approved E-test (0.5 McF inoculum) results are reported as mg/L vancomycin, S = sensitive, I = intermediate. Values for MRSA and in vitro strains were determined by us, for VISA are as reported by NARSA (www.narsa.net).
^{c}Agar screen results are reported as: CG, confluent growth; IC, individual colonies; NG, no growth. Hours of incubation are listed in brackets, data is as reported by NARSA (www.narsa.net).
^{d}MLST (multilocus sequence typing) was either carried out or confirmed here; others are from various literature sources.

### Protein Sample Preparation

Overnight cultures of strains were diluted 1/100 in tryptic soy broth containing 3 mg/L vancomycin for VISA strains and grown to mid-log phase. 20 OD units of cells were harvested, washed 1 time in PBS, and incubated in 220 μL 20 mM Tris-HCl, pH 7.5, 50 L 1 mg/mL lysostaphin, 3 μL protease inhibitor cocktail, and 6 μL DNase for 35 min at 37° C. (approximately 300 μL final volume). Subsequently, 700 μL of 2D lysis buffer (7M urea, 2M thiourea, 3% CHAPS, 20 mM DTT, 5 mM TCEP, 0.5% IPG buffer pH 4-7, 0.25% IPG buffer pH 3-10) was added, samples were vortexed and incubated at RT for 2 h. Samples were centrifuged at maximum speed in a microcentrifuge for 2 min to remove insoluble material, and protein was quantitated using the 2D Quant Kit (Amersham). Protein was precipitated using the 2D Cleanup Kit (Amersham) and resolubilized in 2D lysis buffer.

### Two-Dimensional Gel Electrophoresis (2DE)

In the first dimension, 150 μg protein samples were run on 24 cm Immobiline DryStrips (Amersham) on an IPGphorII IEF system (Amersham) as recommended by the manufacturer. Strips were equilibrated in equilibration buffer (50 mM Tris-Cl pH 8.8, 6 M urea, 30% glycerol, 2% SDS, trace of bromophenol blue) containing 10 mg/ml DTT for 15 minutes and then in equilibration buffer containing 25 mg/ml iodoacetamide for 15 minutes, and sealed to 12% acrylamide gels using 0.5% agarose in standard Tris-glycine electrophoresis buffer. Second dimension SDS-PAGE were run in an EttanDALT apparatus (Amersham) at 40 mA/gel and 15° C. until the tracking dye was run off the gel. Proteins were visualised by Sypro Ruby fluorescence (Invitrogen). Gels were fixed overnight in 40% methanol, 7% acetic acid, stained for a minimum of 5 h, and then destained in 10% methanol, 7% acetic acid for 3×1 h. Gels were imaged with the ProXpress CCD camera-based scanner (Perkin Elmer) at 100 μm resolution using 480 nm excitation and 620 nm emission filters. For each strain, 2D gels of 4 independent samples were analysed using Progenesis Discovery v. 2004 (Nonlinear Dynamics).

### Mass Spectrometry

Gel plugs containing the proteins of interest were excised using a ProXcision robot (Perkin Elmer) and sent for LC/MS/MS analyses (Eastern Quebec Proteomics Centre, Centre Hospitalier de l'Université Laval, Quebec). Gel plugs were placed in 96-well plates and then washed with water. Tryptic digestions were performed on a MassPrep liquid handling robot (Micromass) according to the manufacturer's specifications and using sequencing grade modified trypsin (Promega). After extraction from the gel into 50% acetonitrile/water, peptides were lyophilized in a speed vacuum and resuspended in 10 μl of 0.1% formic acid solution. Peptide MS/MS spectra were obtained by capillary liquid chromatography coupled to an LCQ DecaXP (ThermoFinnigan, San Jose, Calif.) quadrupole ion trap mass spectrometer with a nanospray interface as described previously [38]. Resulting MS/MS spectra were interpreted using MASCOT [46] and searched against bacterial proteins in the UniRef100 database. Carbamidomethylation of cysteine and partial oxidation of methionine, 2 missed cleavages, and an error tolerance of 2.0 Da for peptides and 0.5 Da for fragments were considered in the search. A peptide was considered a good match if it produced a MASCOT score greater than 35, the cutoff calculated by the software as indicating identity or extensive homology at p<0.05. Each peptide identification was confirmed by manual inspection of the spectrum.

### RNA Sample Preparation

Overnight cultures of strains were diluted 1/100 in tryptic soy broth containing either 0 (for all strains), 1 (for selected MRSA), or 3 (for selected VISA) mg/L vancomycin and grown to mid-log phase. Cells from 1 mL culture were harvested and treated with RNAProtect (Qiagen). Cells were subsequently treated with lysostaphin for 30 min at 37° C., and total RNA was isolated from each sample and each condition using the RNeasy Kit (Qiagen). Samples were then treated with Turbo DNA-free (Ambion, Austin, Tex.) to remove any contaminating genomic DNA. RNA quantity and quality was assessed using an Agilent Technologies 2100 bioanalyzer and RNA 6000 Nano LabChip kit (Agilent, Mountain View, Calif.). Complementary DNA (cDNA) was generated from 688 ng of total RNA using a random primer hexamer following the protocol for Superscript II (Invitrogen, Carlsbad, Calif.) in a 20 μL volume.

Real-Time Quantitative RT-PCR (qRT-PCR)

Equal amounts (34 ng) of cDNA were run in triplicate and amplified in a 15 μl reaction containing 7.5 μL of 2× Universal PCR Master Mix (Applied Biosystems, Foster City, Calif.), 10 nM of Z-tailed forward primer, 100 nM of reverse primer, 100 nM of Amplifluor Uniprimer probe (Chemicon, Temecula, Calif.), and 1 μL of DNA target. Moreover, no-template controls were used as recommended. The mixture was incubated at 50° C. for 2 min, at 95° C. for 4 min, and then cycled at 95° C. for 15 sec and at 55° C. for 40 sec 55 times using the Applied Biosystems Prism 7900 Sequence Detector. Amplification efficiencies were validated and normalized to expression of the 16S rRNA gene as a standard, and quantity of target and standard genes were calculated according to a standard curve. Primers were designed using Primer Express 2.0 (Applied Biosystems) and their sequence data are the following: for SAV2095, forward, 5-Ztail-GAAGTTGAAGCACCACAAAATGC-3, reverse, 5-TGTTGATGCTTGTGGTTGTTGAG-3; and for 16S rRNA, forward, 5-Ztail-TCGTGTCGTGAGATGTTGGG-3, reverse, 5-GCTTAAGGGTTGCGCTCGT-3. Amplicons were detected using the Amplifluor™ UniPrimerr™ system where forward primers used contained the 5′ Z sequence ACTGAACCTGACCGTACA. As it is appreciated by a person skilled in the art, it is to be understood herein that other primers may be used for amplifying and detecting SAV2095 without departing from the scope of the invention. A person skilled in the art knows how to design primers for any given amplification assay. For example, in a multiplex assay, primers would need to have similar melting temperatures and low complementarity, etc.

### VISA Quantitation in Blood

Overnight cultures were diluted 1/50 in brain-heart infusion broth and grown to mid-log phase. Bacteria (about 10⁷) and 0.5 mL human blood were added to tubes containing Universal Lysis Beads. Samples were centrifuged at 13000 rpm in a microcentrifuge for 1 min and resuspended in 1 mL TE buffer (10 mM Tris, 1 mM EDTA) twice. Pellets were treated with 0.5 mL RNAprotect (Qiagen) for 5 min and centrifuged again. Two more TE washes were carried out. After the final wash, the remaining few uL of TE was removed. Samples were resuspended in 50 uL of 5xTE and vortexed at maximum speed on a bench-top vortex for 5 min. Samples were spun briefly to collect the contents and incubated at 95° C. for 2 min. After centrifugation at 13000 rpm for 1 min, the supernatant was subjected to a clean-up step using the RNeasy Kit (Qiagen) and eluted in 30 uL RNase-free water. This was treated with TurboDNA-free (Ambion) to remove contaminating DNA. cDNA was generated using random hexamer primers and 8 uL of RNA following the protocol for Superscript II (Invitrogen) in a 20 uL volume. Controls containing no RT enzyme were made for each sample. Serial dilutions of one sample (4× 1/10) were made to be used for standard curve generation. Real-time PCR was carried out in a 25 uL reaction volume using iQ SYBR Green Supermix (Bio-Rad) in a Rotor-Gene apparatus (Corbett Research) according to the manufacturers' directions, using 1 uL of cDNA (either with or without RT) and 1 uL each of 10 uM primer. A no template control was also run. Amplification conditions were as follows: initial denaturation at 95° C. for 5 min, 40-45 cycles of 95° C. 10 sec, annealing 15 sec, 72° C. 20 sec, then a melt from 72° C. to 95° C. Annealing temperature was 56° C. for sceD and 53° C. for 16S. Primers were as follows: sceD forward, JD83, 5′-AAT CCA ACA TCA GGT GCA GC-3′; sceD reverse, JD84, 5′-GCA GTA ACC CM TGT CCA GC-3′; 16S forward, JD79, 5′-ACG AGC GCA ACC CTT AAG-3′; 16S reverse, JD80, 5′-TTC GCT GCC CTT TGT ATT G-3′. Relative expression levels were calculated according to a standard curve and sceD expression was normalized to the expression of the 16S rRNA gene to control for varying amounts of initial RNA.

### Electron Microscopy

Bacterial colonies from a fresh overnight plate were resuspended in PBS and fixed with 2% EM-grade glutaraldehyde, 0.1 M cacodylate pH 7.4. Embedding, thin sectioning, and heavy-metal staining were carried out using standard techniques. Grids were visualized on a JEOL 1010 transmission electron microscope at 80 kV under 150000× magnification. Cell wall thicknesses were measured with the help of Digital Micrograph software (Gatan Inc.). For each strain, the cell wall thickness at 3 points of 10 representative cells were measured and averaged.

### Example 1

### Identification of SAV2095 in 2D Gel Comparisons of CMRSA-2 and Mu50

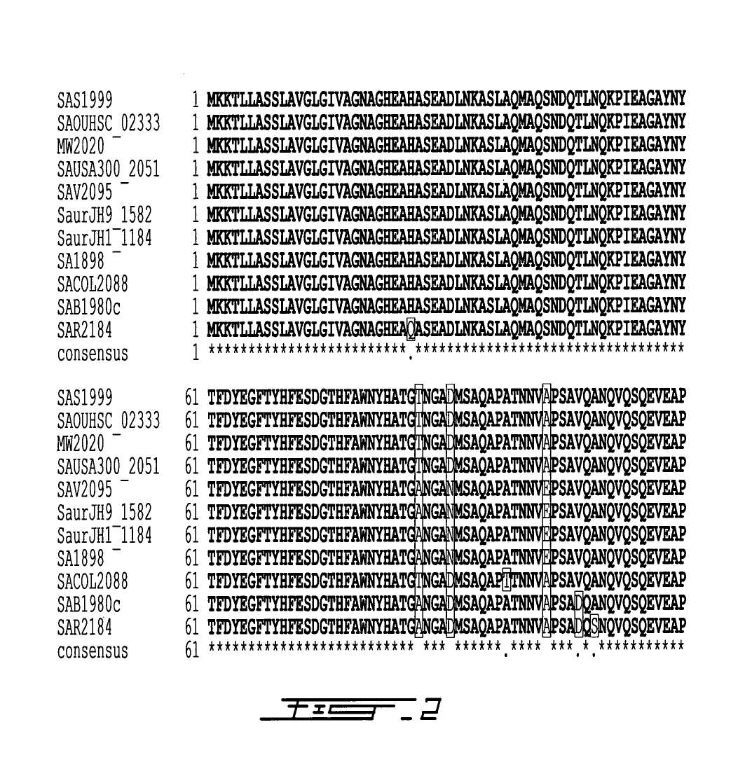

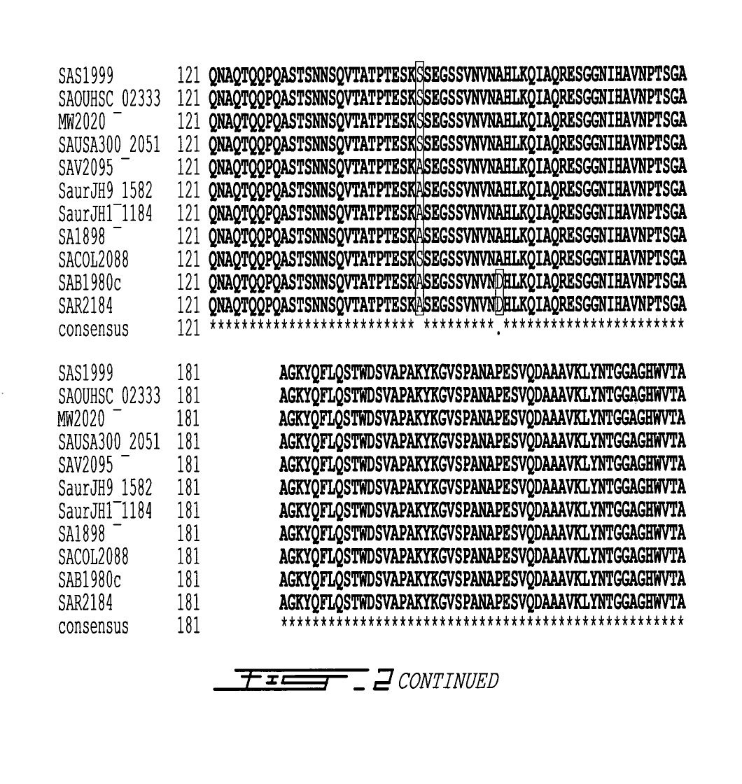

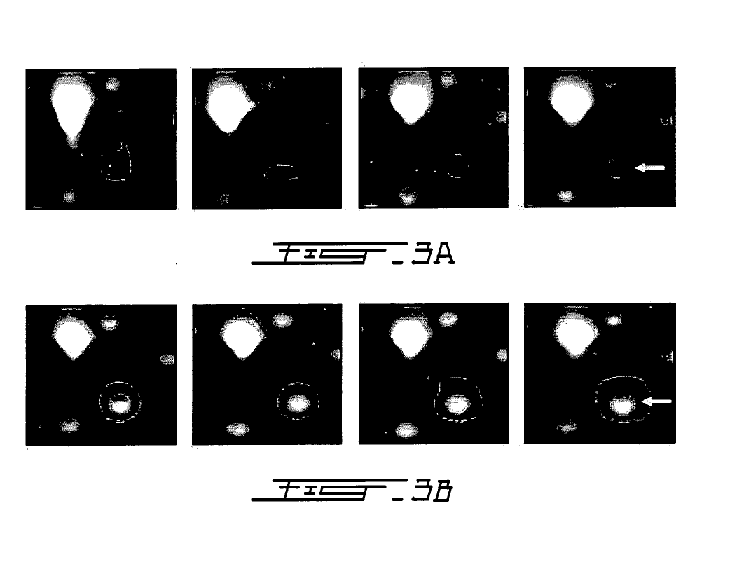

A number of differentially-expressed spots were found throughout this analysis. Among these was a highly-overexpressed spot (expression ratio in Mu50/CMRSA-2=23.5, p<0.02 was subsequently analyzed). Subsequent analysis by tandem mass spectrometry indicated that this spot contained the protein SAV2095, a protein of unknown function with homology to SceD, with a MASCOT score of 256 and protein coverage of 28% (MS#2800). In a second, independent, 2D gel analysis, this spot was again identified (expression ratio in Mu50/CMRSA-2=19.5, p<0.004) with a MASCOT score of 591 and a protein coverage of 34% (MS#5951) as shown in FIG. 3. Further 2D gel analysis of protein extracts from a number of MRSA and hVISA/VISA isolates shows overexpression in all VISA in contrast to MRSA strains suggesting that SAV2095 protein product is generally increased in VISA strains (FIG. 4.)

### Example 2

### Expression of SAV2095 Homologue mRNA in Various MRSA Versus VISA Strains

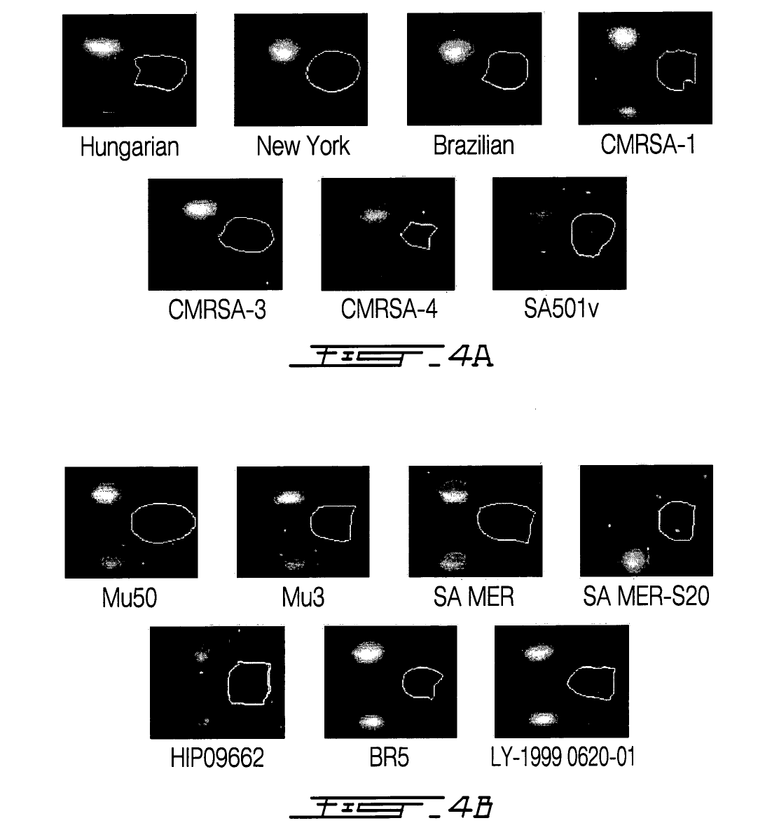

The expression levels of the SAV2095 homologue mRNA were measured by qRT-PCR in a number of MRSA and VISA strains (FIG. 5). As seen in Table 1, these strains cover several genetic backgrounds, come from several countries, and display a range of vancomycin MICs. SAV2095 measurements were normalised to 16S rRNA levels, since expression of this gene was not found to vary with resistance phenotype or vancomycin treatment although other internal controls may be used. Overall, the difference in SAV2095 expression level between MRSA and VISA was highly significant (p<0.0001). Individually, VISA strains showed significantly higher expression levels than almost all MRSA tested. CMRSA-1 may be an hV1SA as described below.

### Example 3

### Electron Microscopy Analysis of MRSA and in vitro-Selected Mutants

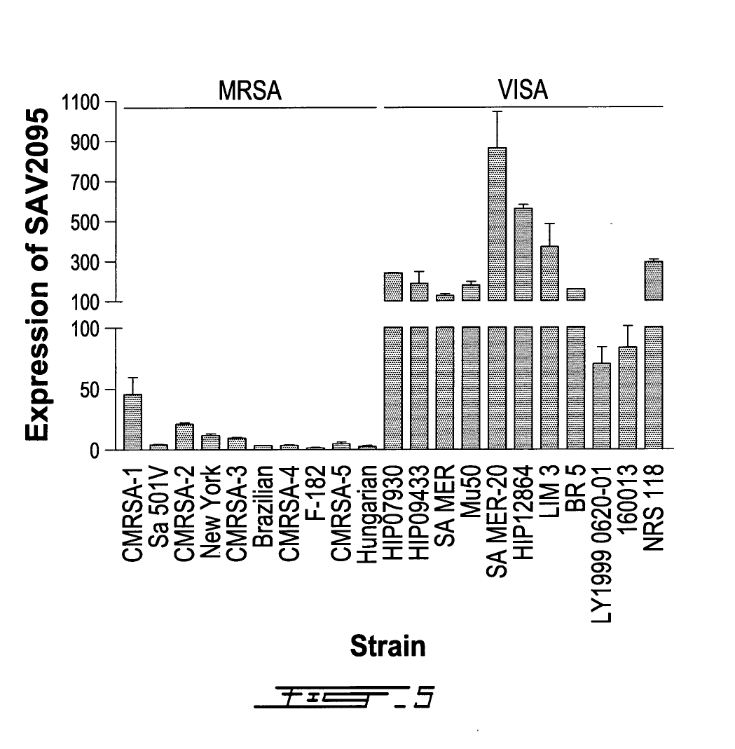

Electron microscopy (FIG. 6) shows that strains selected in vitro for intermediate levels of vancomycin resistance show thickened cell walls compared to their parent VSSA strains (panels B and C) providing evidence of the hVISA or VISA phenotype of these strains. Interestingly, the CMRSA-1 strain also displays a thickened cell wall (panel A), which increases further in thickness with increasing vancomycin resistance (strain CIV8). Combining all the information obtained for CMRSA-1 here with Etest results described in Table 1, a hVISA phenotype appears to be more appropriate than a VSSA phenotype for this strain, which is further exemplified below.

### Example 4

### Expression of SAV2095 Homologue mRNA in Various MRSA Versus hVISA and VISA Strains

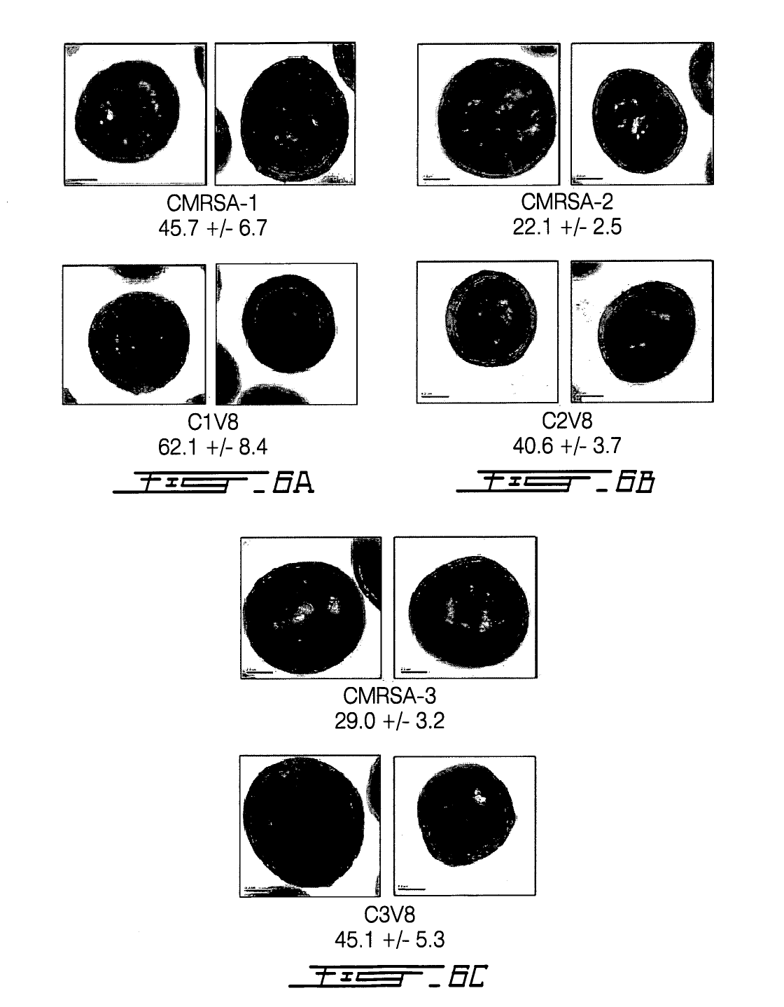

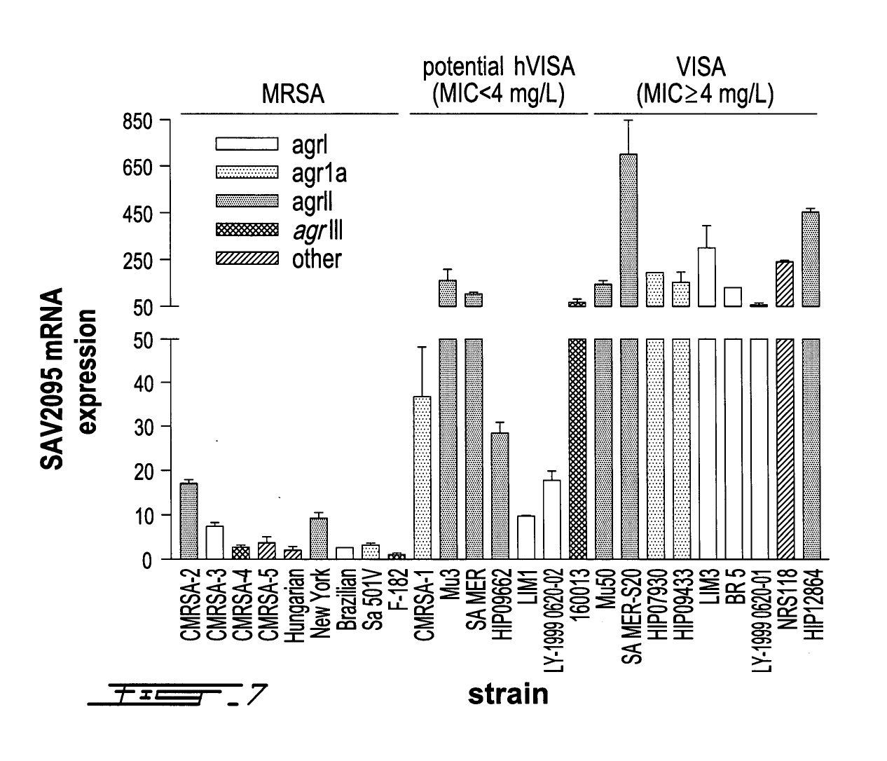

The expression levels of the SAV2095 homologue mRNA in the absence of Vn were measured by qRT-PCR in a number of MRSA and VISA strains, including hVISA (FIG. 7). As seen in Table 1, all strains cover several genetic backgrounds, come from several countries, and display a range of vancomycin MICs. Individually, all strains with Vn MIC≧4 mg/L by standard Etest showed higher expression levels than all MRSA tested. High levels of SAV2095 mRNA expression were seen in isolates of all agr and MLST types, showing that induction of this gene is a general trait of VISA strains and is independent of genetic background. Six strains tested in this study show Vn MIC<4 mg/L using Clinical and Laboratory Standards Institute (CLSI)-approved methods, but appear to have intermediate levels of resistance by macromethod Etest using a 2 McFarland inoculum (Table 1). A MIC≧4 mg/L by macromethod Etest is indicative of hVISA/VISA. We tested SAV2095 levels in 6 ‘potential’ hVISA/VISA strains (CLSI Etest<4 mg/L, macromethod Etest≧4 mg/L). Three of these strains (i.e. Mu3, SA MER and 160013) showed levels of SAV2095 expression indistinguishable from those of more highly-resistant strains, while the others (LIM1, LY-1999 0620-02 and HIP09662) did not. LIM1 (NRS35) and LY-1999 0620-02 (NRS64), did not show higher levels of expression of particular gene products increased in some other VISA strains it therefore seems possible that LIM1 and LY-1999 0620-02 might express resistance mechanisms distinct from those of other strains or may not be hVISA isolates. SAV2095 levels may therefore be useful for detecting at least some hVISA isolates.

By macromethod Etest, CMRSA-1 showed individual colony growth after 48 h incubation at 8 mg/L Vn (Table 1) and thickened cell walls by electron microscopy (FIG. 6). Interestingly, CMRSA-1 also showed disproportionately high levels of SAV2095 expression with respect to the other MRSA strains (FIG. 5, 7). CMRSA-1 may be a previously unrecognized hVISA strain.

### Example 5

### SAV2095 mRNA Quantitation Directly from Blood

Currently, detection of VISA requires initial culture, isolation, and the vancomycin agar screen test described in the introduction. Together, this takes several days, during which time patient treatment is ineffective. The identification of a molecular marker to detect VISA strains could rapidly and accurately determine whether an isolate displays clinically-relevant levels of resistance to vancomycin, in the absence of subculture is overly advantageous over the currently available kits. Such a kit could help to direct the therapeutic approach, and improve the outcome of patients infected with VISA. This kit could take several forms, for example as an antibody-based ELISA, a mass spectrometry-based technique such as multiple reaction monitoring, or a real-time RT-PCR based test.

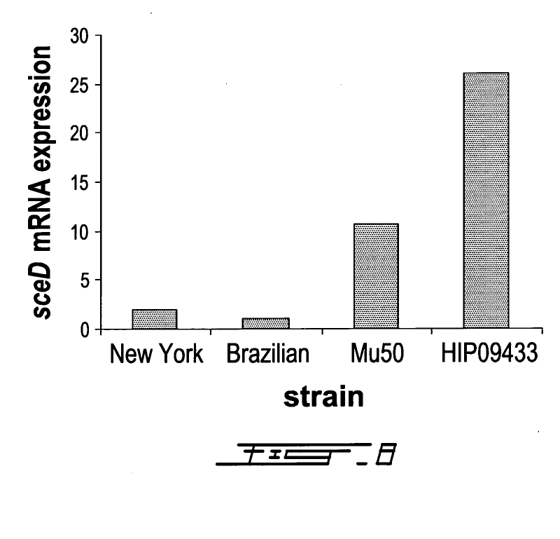

To demonstrate that difference in SAV2095 (sceD) expression could be measured from clinical samples of biological fluids, human blood was spiked with MRSA and VISA. Such samples were then subjected to bacterial RNA purification and sceD quantitation by real-time RT-PCR. As shown in FIG. 8 the increased levels of sceD mRNA in VISA strains compared to MRSA could clearly be measured.

### Example 6

### Expression of SAV2095 in Response to Growth in Sub-Inhibitory Concentrations of Vancomycin

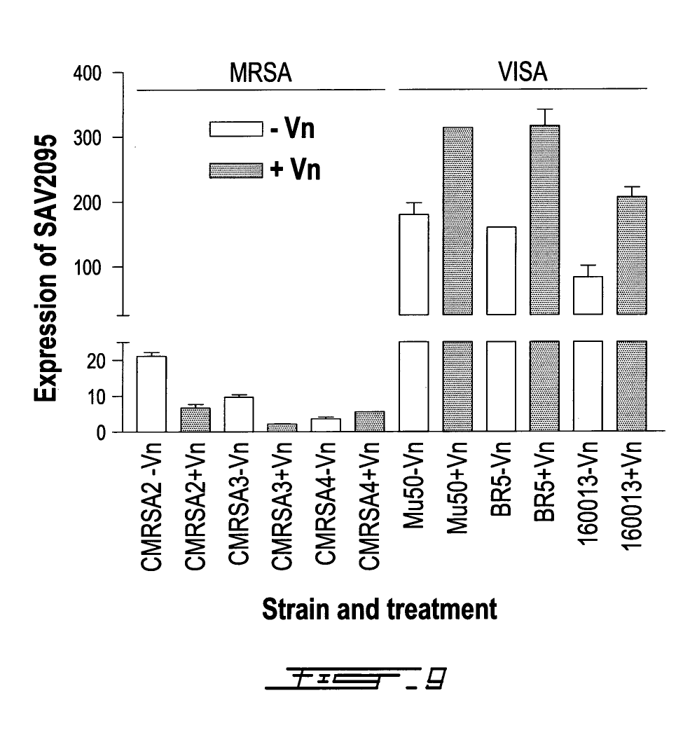

In addition, we found that growth in the presence of low levels of Vn increased expression of SAV2095 in VISA strains of different backgrounds but not in MRSA (p<0.05 in VISA; FIG. 9).

This is the first demonstration that SAV2095 levels are consistently increased in numerous VISA versus vancomycin-sensitive MRSA strains from a variety of genetic backgrounds, and that growth in the presence of subinhibitory levels of vancomycin further increases the expression of this gene in VISA. This suggests that the differences seen in FIG. 7 could be amplified even further if samples were grown in the presence of subinhibitory concentrations of vancomycin (e.g. 1 mg/L). It could also present another characteristic that differentiates VISA from MRSA for diagnostic purposes.

### Example 7

### Expression of SAV2095 in Isogenic (Closely Genetically Related) Displaying Increasing Levels of Vancomycin Resistance

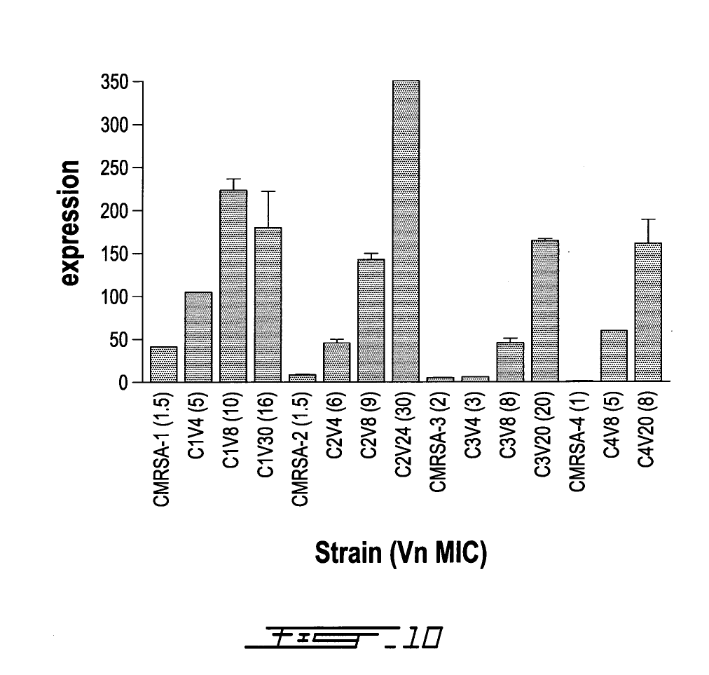

To further clarify the effects of resistance level on SAV2095 expression, levels of SAV2095 mRNA were determined for series of in vitro-selected strains derived from MRSA (VSSA) parent strains of 4 distinct genetic backgrounds (FIG. 10). Expression level increased with increasing resistance in all cases. Further increases in expression level were also seen between related clinical isolates with different levels of resistance. This suggests that monitoring SAV2095 expression over the course of treatment could also show correlations with the emergence of resistance.

### Example 8

### Identification of a VISA Strain in a Patient

A biological sample is obtained from a patient suspected of having a bacterial infection in order to determine the vancomycin susceptibility or resistance profile of the strain causing the infection. The sample may be subculture in absence of vancomycin or used directly for analysis. The biological sample is analyzed for the presence or absence of a VISA strain by detecting the expression products of SEQ ID NO.:1, portion, homolog or complement thereof in the sample. To that effect, RT-PCR is performed using primers selected to amplifya region of at least 15 nucleotides of SEQ ID NO.:1. Primers may be degenerated to allow amplification of any of SEQ ID NO.:1 homologs. The expression product is compared to a reference value. If the expression product of SEQ ID NO.:1 portion, homolog or complement thereof is higher than a negative control, for example, a MRSA strain, the patient is suspected of having a VISA strain and a non-vancomycin treatment is prescribed. The treatment has therefore a higher chance of success. If the expression product of SEQ ID NO.:1 portion, homolog or complement thereof is substantially at the same level as a positive control, for example a known VISA strain, a non-vancomycin treatment is prescribed. The patient may be put in isolation. When the expression product of SEQ ID NO.:1, portion, homolog or complement thereof is at the same level as a negative control, for example a non-VISA strain such as a MRSA strain, a vancomycin treatment is prescribed and has a good probability of success. The determination of vancomycin resistance profile prior to or during treatment may allow for better control of vancomycin resistance development. Alternatively, the protein encoded by SEQ ID NO.:1 is detected using an antibody and the level of protein is compared to that of a (negative, positive) reference value.

In the event that a patient is first diagnosed not to carry a VISA strain and prescribed a vancomycin drug treatment, the progression of the treatment and the emergence of vancomycin resistance is monitored by detecting the expression product of SEQ ID NO.:1, portion, homolog or complement thereof over the course of the treatment, at whichever interval the medical practitioner finds appropriate. If the practitioner observes that the expression product or SEQ ID NO.:1 portion, homolog or complement thereof increases over time, especially at a level comparable to a known VISA strain, the practitioner may wish to stop treatment and change for a non-vancomycin drug to ensure better and higher probability of effective treatment.

Although the present invention has been described hereinabove by way of exemplary embodiments, it can be modified without departing from the spirit, scope and the nature of the invention.

The present description refers to a number of documents, the content of which is herein incorporated by reference in their entirety.

### REFERENCES

1. Hiramatsu, K., H. Hanaki, T. Ino, K. Yabuta, T. Oguri and F. C. Tenover. 1997. Methicillin-resistant Staphylococcus aureus clinical strain with reduced vancomycin susceptibility. Journal of Antimicrobial Chemotherapy, 40(1): 135-6.
2. Wong, S. S., P. L. Ho, P. C. Woo and K. Y. Yuen. 1999. Bacteremia caused by staphylococci with inducible vancomycin heteroresistance. Clinical Infectious Diseases, 29(4): 760-7.
3. Smith, T. L., M. L. Pearson, K. R. Wilcox, C. Cruz, M. V. Lancaster, B. Robinson-Dunn, et al. 1999. Emergence of vancomycin resistance in Staphylococcus aureus. Glycopeptide-lntermediate Staphylococcus aureus Working Group. New England Journal of Medicine, 340(7): 493-501.
4. Ploy, M. C., C. Grelaud, C. Martin, L. de Lumley and F. Denis. 1998. First clinical isolate of vancomycin-intermediate Staphylococcus aureus in a French hospital. Lancet, 351(9110): 1212.
5. Mainardi, J. L., D. M. Shlaes, R. V. Goering, J. H. Shlaes, J. F. Acar and F. W. Goldstein. 1995. Decreased teicoplanin susceptibility of methicillin-resistant strains of Staphylococcus aureus. Journal of Infectious Diseases, 171(6): 1646-50.
6. Howe, R. A., K. E. Bowker, T. R. Walsh, T. G. Feest and A. P. MacGowan. 1998. Vancomycin-resistant Staphylococcus aureus. Lancet, 351(9102): 602.
7. Bobin-Dubreux, S., M. E. Reverdy, C. Nervi, M. Rougier, A. Bolmstrom, F. Vandenesch, and J. Etienne. 2001. Clinical isolate of vancomycin-heterointermediate Staphylococcus aureus susceptible to methicillin and in vitro selection of a vancomycin-resistant derivative. Antimicrob Agents Chemother, 45(1): 349-52.
8. Palazzo, I. C., M. L. Araujo and A. L. Darini. 2005. First report of vancomycin-resistant staphylococci isolated from healthy carriers in Brazil. J Clin Microbiol, 43(1): 179-85.
9. Pootoolal, J., J. Neu and G. D. Wright. 2002. Glycopeptide antibiotic resistance. Annual Review of Pharmacology and Toxicology, 42: 381-408.
10. 2002. Staphylococcus aureus resistant to vancomycin—United States, 2002. MMWR. Morbidity and Mortality Weekly Report, 51(26): 565-7.
11. 2002. Vancomycin-resistant Staphylococcus aureus—Pennsylvania, 2002. MMWR. Morbidity and Mortality Weekly Report, 51(40): 902.
12. Chaterjee, A. N. and H. R. Perkins. 1966. Compounds formed between nucleotides related to the biosynthesis of bacterial cell wall and vancomycin. Biochemical and Biophysical Research Communications, 24: 489-494.
13. Ishino, F., W. Park, S. Tomioka, S. Tamaki, I. Takase, K. Kunugita, et al. 1986. Peptidoglycan synthetic activities in membranes of Escherichia coli caused by overproduction of penicillin-binding protein 2 and rodA protein. Journal of Biological Chemistry, 261(15): 7024-31.
14. Cui, L., X. Ma, K. Sato, K. Okuma, F. C. Tenover, E. M. Mamizuka, et al. 2003. Cell wall thickening is a common feature of vancomycin resistance in Staphylococcus aureus. Journal of Clinical Microbiology, 41(1): 5-14.
15. Cui, L., H. Murakami, K. Kuwahara-Arai, H. Hanaki and K. Hiramatsu. 2000. Contribution of a thickened cell wall and its glutamine nonamidated component to the vancomycin resistance expressed by Staphylococcus aureus Mu50. Antimicrobial Agents and Chemotherapy, 44(9): 2276-85.
16. Sieradzki, K. and A. Tomasz. 1997. Inhibition of cell wall turnover and autolysis by vancomycin in a highly vancomycin-resistant mutant of Staphylococcus aureus. Journal of Bacteriology, 179(8): 2557-66.
17. Hanaki, H., K. Kuwahara-Arai, S. Boyle-Vavra, R. S. Daum, H. Labischinski and K. Hiramatsu. 1998. Activated cell-wall synthesis is associated with vancomycin resistance in methicillin-resistant Staphylococcus aureus clinical strains Mu3 and Mu50. Journal of Antimicrobial Chemotherapy, 42(2): 199-209.
18. Cui, L., A. Iwamoto, J. Q. Lian, H. M. Neoh, T. Maruyama, Y. Horikawa, and K. Hiramatsu. 2006. Novel mechanism of antibiotic resistance originating in vancomycin-intermediate Staphylococcus aureus. Antimicrob Agents Chemother, 50(2): 428-38.
19. Komatsuzawa, H., K. Ohta, S. Yamada, K. Ehlert, H. Labischinski, J. Kajimura, T. Fujiwara, and M. Sugai. 2002. Increased glycan chain length distribution and decreased susceptibility to moenomycin in a vancomycin-resistant Staphylococcus aureus mutant. Antimicrobial Agents and Chemotherapy, 46(1): 75-81.
20. Hanaki, H., H. Labischinski, Y. Inaba, N. Kondo, H. Murakami and K. Hiramatsu. 1998. Increase in glutamine-non-amidated muropeptides in the peptidoglycan of vancomycin-resistant Staphylococcus aureus strain Mu50. Journal of Antimicrobial Chemotherapy, 42(3): 315-20.
21. Stranden, A. M., M. Roos and B. Berger-Bachi. 1996. Glutamine synthetase and heteroresistance in methicillin-resistant Staphylococcus aureus. Microbial Drug Resistance, 2(2): 201-7.
22. Moreira, B., S. Boyle-Vavra, B. L. deJonge and R.S. Daum. 1997. Increased production of penicillin-binding protein 2, increased detection of other penicillin-binding proteins, and decreased coagulase activity associated with glycopeptide resistance in Staphylococcus aureus. Antimicrobial Agents and Chemotherapy, 41(8): 1788-93.
23. Reipert, A., K. Ehlert, T. Kast and G. Bierbaum. 2003. Morphological and genetic differences in two isogenic Staphylococcus aureus strains with decreased susceptibilities to vancomycin. Antimicrobial Agents and Chemotherapy, 47(2): 568-76.
24. Pfeltz, R. F., V. K. Singh, J. L. Schmidt, M. A. Batten, C. S. Baranyk, M. J. Nadakavukaren, R. K. Jayaswal, and B. J. Wilkinson. 2000. Characterization of passage-selected vancomycin-resistant Staphylococcus aureus strains of diverse parental backgrounds. Antimicrobial Agents and Chemotherapy, 44(2): 294-303.
25. Daum, R. S., S. Gupta, R. Sabbagh and W. M. Milewski. 1992. Characterization of Staphylococcus aureus isolates with decreased susceptibility to vancomycin and teicoplanin: isolation and purification of a constitutively produced protein associated with decreased susceptibility. Journal of Infectious Diseases, 166(5): 1066-72.
26. Shlaes, D. M., J. H. Shlaes, S. Vincent, L. Etter, P. D. Fey and R. V. Goering. 1993. Teicoplanin-resistant Staphylococcus aureus expresses a novel membrane protein and increases expression of penicillin-binding protein 2 complex. Antimicrobial Agents and Chemotherapy, 37(11): 2432-7.
27. Boyle-Vavra, S., R. B. Carey and R. S. Daum. 2001. Development of vancomycin and lysostaphin resistance in a methicillin-resistant Staphylococcus aureus isolate. Journal of Antimicrobial Chemotherapy, 48(5): 617-25.
28. Sieradzki, K. and A. Tomasz. 1999. Gradual alterations in cell wall structure and metabolism in vancomycin-resistant mutants of Staphylococcus aureus. Journal of Bacteriology, 181(24): 7566-70.
29. Wyke, A. W., J. B. Ward, M. V. Hayes and N. A. Curtis. 1981. A role in vivo for penicillin-binding protein-4 of Staphylococcus aureus. European Journal of Biochemistry, 119(2): 389-93.
30. Finan, J. E., G. L. Archer, M. J. Pucci and M. W. Climo. 2001. Role of penicillin-binding protein 4 in expression of vancomycin resistance among clinical isolates of oxacillin-resistant Staphylococcus aureus. Antimicrobial Agents and Chemotherapy, 45(11): 3070-5.
31. Mongodin, E., J. Finan, M. W. Climo, A. Rosato, S. Gill and G. L. Archer. 2003. Microarray transcription analysis of clinical Staphylococcus aureus isolates resistant to vancomycin. Journal of Bacteriology, 185(15): 4638-43.
32. Pieper, R., C. L. Gatlin-Bunai, E. F. Mongodin, P. P. Parmar, S. T. Huang, D. J. Clark, R. D. Fleischmann, S. R. Gill, and S. N. Peterson. 2006. Comparative proteomic analysis of Staphylococcus aureus strains with differences in resistance to the cell wall-targeting antibiotic vancomycin. Proteomics, 6(15): 4246-58.
33. McAleese, F., S. W. Wu, K. Sieradzki, P. Dunman, E. Murphy, S. Projan, and A. Tomasz. 2006. Overexpression of genes of the cell wall stimulon in clinical isolates of Staphylococcus aureus exhibiting vancomycin-intermediate-S. aureus-type resistance to vancomycin. J Bacteriol, 188(3): 1120-33.
34. Walsh, T. R. and R. A. Howe. 2002. The prevalence and mechanisms of vancomycin resistance in Staphylococcus aureus. Annual Review of Microbiology, 56: 657-75.
35. Hiramatsu, K., N. Aritaka, H. Hanaki, S. Kawasaki, Y. Hosoda, S. Hori, Y. Fukuchi, and I. Kobayashi. 1997. Dissemination in Japanese hospitals of strains of Staphylococcus aureus heterogeneously resistant to vancomycin. Lancet, 350(9092): 1670-3.
36. Nishi, H., H. Komatsuzawa, T. Fujiwara, N. McCallum and M. Sugai. 2004. Reduced content of lysyl-phosphatidylglycerol in the cytoplasmic membrane affects susceptibility to moenomycin, as well as vancomycin, gentamicin, and antimicrobial peptides, in Staphylococcus aureus. Antimicrob Agents Chemother, 48(12): 4800-7.
37. Ruzin, A., A. Severin, S. L. Moghazeh, J. Etienne, P. A. Bradford, S. J. Projan, and D. M. Shlaes. 2003. Inactivation of mprF affects vancomycin susceptibility in Staphylococcus aureus. Biochimica et Biophysica Acta, 1621(2): 117-21.
38. Brandenberger, M., M. Tschierske, P. Giachino, A. Wada and B. Berger-Bachi. 2000. Inactivation of a novel three-cistronic operon tcaR-tcaA-tcaB increases teicoplanin resistance in Staphylococcus aureus. Biochimica et Biophysica Acta, 1523(2-3): 135-9.
39. Maki, H., N. McCallum, M. Bischoff, A. Wada and B. Berger-Bachi. 2004. tcaA inactivation increases glycopeptide resistance in Staphylococcus aureus. Antimicrob Agents Chemother, 48(6): 1953-9.
40. Wootton, M., A. P. Macgowan and T. R. Walsh. 2005. Expression of tcaA and mprF and glycopeptide resistance in clinical glycopeptide-intermediate Staphylococcus aureus (GISA) and heteroGISA strains. Biochim Biophys Acta, 1726(3): 326-7.
41. Papakyriacou, H., D. Vaz, A. Simor, M. Louie and M. J. McGavin. 2000. Molecular analysis of the accessory gene regulator (agr) locus and balance of virulence factor expression in epidemic methicillin-resistant Staphylococcus aureus. J Infect Dis, 181(3): 990-1000.
42. Vandesompele et al 2002 Genome biology 3:0034.1-0034.
43. Cronin et al 2004 Clin Chem 50:1464-1471.

1. A method for identifying a vancomycin-intermediate Staphylococcus aureus (VISA) strain in a sample, said method comprising
measuring an expression level of
a) an expression product of SEQ ID NO.:1 or of a SEQ ID NO.:1 homolog,
b) a complement of a), or
c) a portion of a) or b), and
comparing the expression level with a reference value.

2. The method of claim 1 wherein the sample is a biological sample.

3. The method of claim 1, wherein the reference value is obtained from at least one non-VISA strain.

4. The method of claim 3 wherein the non-VISA strain is a MRSA strain.

5. The method of claim 1, wherein the reference value is from at least one VISA strain.

6. The method of claim 1, wherein the expression product is RNA.

7. The method of claim 1, wherein the expression product is a polypeptide.

8. The method of claim 7 wherein the polypeptide is measured by immunoassay.

9. A diagnostic kit for identifying a vancomycin-intermediate Staphylococcus aureus (VISA) in a sample, said kit comprising a reagent for measuring the expression level of
a) an expression product of SEQ ID NO.:1 or a SEQ ID NO.:1 homolog,
b) a complement of a), or
c) a portion of a) or b).

10. The kit of claim 9 wherein the reagent is selected from the group consisting of a sequence substantially complementary to SEQ ID NO.:1, a sequence substantially complementary to a SEQ ID NO.:1 homolog, a sequence substantially complementary to a complement of SEQ ID NO.:1, a sequence substantially complementary to a complement of a homolog of SEQ ID NO.:1, a sequence substantially complementary to a portion of SEQ ID NO.:1 and a sequence substantially complementary to a portion of a homolog of SEQ ID NO.:1.

11. The kit of claim 9 wherein the reagent is selected from the group consisting of an antibody, an antibody binding fragment and aptamers.

12. A method for facilitating the development of an individualized treatment regimen for a patient suspected of having an infection or having an infection in need of treatment, said method comprising
measuring an expression level of
a) an expression product of SEQ ID NO.:1 or of a SEQ ID NO.:1 homolog,
b) a complement of a), or;
c) a portion of a) or b),
comparing the expression level with a reference value, and
administering a vancomycin or non-vancomycin drug regimen in response to the result obtained.

13. The method of claim 12 wherein said infection is a Staphylococcus aureus infection.

14. The method of claim 13 wherein said infection is a drug resistant Staphylococcus aureus infection.

15. The method of claim 14 wherein the drug is methicillin

16. A method for monitoring the progression of a vancomycin treatment of an infection comprising measuring the expression level of
a) an expression product of SEQ ID NO.:1 or of a SEQ ID NO.:1 homolog,
b) a complement of a), or
c) a portion of a) or b)
over the course of the treatment.

17. The method of claim 16 wherein the infection is a Staphylococcus aureus infection.

18. A method for identifying a Staphylococcus aureus strain in a sample, said method comprising detecting an expression level of
a) an expression product of SEQ ID NO.:1 or of a SEQ ID NO.:1 homolog,
b) a complement of a), or;
c) a portion of a) or b).

19. A method of determining the susceptibility of a Staphylococcus aureus strain to vancomycin, wherein the Staphylococcus aureus strain, when using a standard laboratory test, is characterized as a VISA strain, the method comprising detecting:
a) an expression product of SEQ ID NO.:1 or of a SEQ ID NO.:1 homolog,
b) a complement of a) or
c) a portion of a) or b).

20. The method of claim 19, wherein the Staphylococcus aureus strain is characterized by a MIC of ≧4-16 mg/L when using a standard laboratory test.

21. The method of claim 20, wherein the Staphylococcus aureus strain is characterized by a MIC of <8 mg/L when using a standard laboratory test.

22. The method of claim 20, wherein the Staphylococcus aureus strain is characterized by a MIC of ≧4 mg/L when using a standard laboratory test.

23. The method of claim 21, wherein the Staphylococcus aureus strain is characterized by a MIC of ≧4 mg/L when using a standard laboratory test.
